(19) United States
(12) Patent Application Publication
(10) Pub. No.: US 2023/0414142 A1
(43) Pub. Date: Dec. 28, 2023

(54) METHOD AND APPARATUS FOR ESTIMATING USER'S EMOTIONAL STATE

(71) Applicant: Panasonic Intellectual Property Management Co., Ltd., Osaka (JP)

(72) Inventors: HIROTO YANAGAWA, Osaka (JP); HITOSHI TOMINAGA, Kyoto (JP); YUMIKO OHNO, Osaka (JP); SHUNSUKE IMAI, Osaka (JP); TSUGUHIRO KORENAGA, Osaka (JP); YUKIKO UCHIDA, Kyoto (JP)

(21) Appl. No.: 18/462,449

(22) Filed: Sep. 7, 2023

Related U.S. Application Data
(63) Continuation of application No. PCT/JP2022/007210, filed on Feb. 22, 2022.

(30) Foreign Application Priority Data
Mar. 22, 2021 (JP) 2021-047381

(51) Int. Cl. A61B 5/16; A61B 5/00; A61B 5/026; A61B 5/024

(52) U.S. Cl. CPC A61B 5/165; A61B 5/02405; A61B 5/026; A61B 5/74

(57) ABSTRACT

A method for estimating an emotional state is executed by a computer and includes acquiring an amount of change of a cerebral blood flow of a user from a reference time, acquiring an amount of change of a heart rate of the user from a reference time, and outputting a signal indicating that the user is in an excited state in a case where the amount of change of the cerebral blood flow is smaller than a first threshold value and the amount of change of the heart rate is larger than a second threshold value.

### BACKGROUND

### 1. Technical Field

The present disclosure relates to a method and an apparatus for estimating a user's emotional state.

### 2. Description of the Related Art

In recent years, research and development concerning sensing of a state of a person have been actively conducted. A sensed state of a person is related to a cognitive function such as an emotional state or a thinking state. Every emotion is closely related to human lives, and if an emotional state of a person can be estimated by sensing, an improvement in quality of life is expected.

Japanese Unexamined Patent Application Publication No. 2019-102164 discloses a technique of detecting a degree of excitement of audience on the basis of a sound volume detected by sound collecting devices installed around an event site and controlling lighting on the basis of a result of the detection. By controlling lighting in accordance with a sound volume around the event site, excitement of the audience can be calmed or heightened.

Furthermore, methods for estimating a state such as an emotion of a person by sensing an autonomic nervous system have been developed. For example, there has been an attempt to estimate a state such as an emotion by sensing a human autonomic nervous system by using near-infrared spectroscopy (NIRS). In the near-infrared spectroscopy, a near-infrared ray (also referred to as near-infrared light) that easily passes through a living body is used. Hemoglobin in blood of a living body has such a characteristic that near-infrared light absorption spectrum varies depending on whether the hemoglobin is in an oxygenated state or in a deoxygenated state, and this characteristic is utilized. For example, a state of a cerebral blood flow can be estimated by irradiating a forehead with near-infrared light and detecting the light reflected by the forehead. Furthermore, a state of brain activity such as an emotion can be estimated on the basis of the state of the cerebral blood flow.

International Publication No. 2019/176535, International Publication No. 2018/167854, and Japanese Unexamined Patent Application Publication No. 2012-161558 disclose an example of a technique for estimating a state of brain activity by using near-infrared spectroscopy. International Publication No. 2019/176535 discloses estimating an emotion on the basis of a state of a cerebral blood flow and a state of a facial blood flow. International Publication No. 2018/167854 and Japanese Unexamined Patent Application Publication No. 2012-161558 disclose estimating an emotion on the basis of a cerebral blood flow and a heart rate.

### SUMMARY

One non-limiting and exemplary embodiment provides a novel technique that can estimate an emotional state of a person such as an excited state or a relaxed state with higher accuracy than conventional arts.

In one general aspect, the techniques disclosed here feature a method executed by a computer, the method including: acquiring an amount of change of a cerebral blood flow of a user from a reference time; acquiring an amount of change of a heart rate of the user from a reference time; and outputting a signal indicating that the user is in an excited state in a case where the amount of change of the cerebral blood flow is smaller than a first threshold value and the amount of change of the heart rate is larger than a second threshold value.

According to the aspect of the present disclosure, an emotional state of a person such as an excited state or a relaxed state can be estimated with higher accuracy than conventional arts.

It should be noted that general or specific aspects of the present disclosure may be implemented as a system, an apparatus, a method, an integrated circuit, a computer program, a computer-readable recording medium, or any selective combination thereof. Examples of the computer-readable recording medium include non-volatile recording media such as a compact disc-read only memory (CD-ROM), a digital versatile disc (DVD), and a Blu-ray disc (BD). The apparatus may include one or more apparatuses. In a case where the apparatus includes two or more apparatuses, the two or more apparatuses may be disposed in a single apparatus or may be separately disposed in separate two or more apparatuses. In the specification and claims, the “apparatus” can mean not only a single apparatus, but also a system including apparatuses. Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 illustrates an example of a condition on which it is determined that a user is in an excited state or a relaxed state;

FIG. 2 illustrates another example of a condition on which it is determined that a user is in an excited state or a relaxed state;

FIG. 3 illustrates an example of a configuration of a system for estimating a user's emotional state;

FIG. 4A illustrates an example of a temporal change of an emitted light pulse and temporal changes of an intensity of a surface reflected component and an intensity of an internal scattered component in a reflected light pulse;

FIG. 4B illustrates another example of a temporal change of an emitted light pulse and temporal changes of an intensity of a surface reflected component and an intensity of an internal scattered component in a reflected light pulse;

FIG. 5 illustrates an example of an outline configuration of a single pixel of a light receiving device;

FIG. 6 is a diagram illustrating an example of a configuration of the light receiving device;

FIG. 7 schematically illustrates an example of operation performed within 1 frame;

FIG. 8 is a view for explaining an operation of measuring a cerebral blood flow;

FIG. 9A is a timing diagram illustrating an example of an operation of detecting an internal scattered component;

FIG. 9B is a timing diagram illustrating an example of an operation of detecting a surface reflected component;

FIG. 10 is a flowchart illustrating an outline of an operation of controlling a light emitting device and the light receiving device;

FIG. 11 illustrates an example in which a cerebral blood flow sensor is a contact type NIRS device;

FIG. 12 schematically illustrates an example of a configuration of the contact type NIRS device on a rear side;

FIG. 13 is a flowchart illustrating an example of emotion estimation processing;

FIG. 14 is a flowchart illustrating another example of the emotion estimation processing;

FIG. 15 is a flowchart illustrating still another example of the emotion estimation processing;

FIG. 16 is a flowchart illustrating still another example of the emotion estimation processing;

FIG. 17 is a flowchart illustrating still another example of the emotion estimation processing;

FIG. 18A is a graph illustrating time-series data of a cerebral blood flow amount obtained in a case where subjects listened to exciting music;

FIG. 18B is a graph illustrating time-series data of a cerebral blood flow amount obtained in a case where the subjects listened to relaxing music;

FIG. 18C is a graph illustrating time-series data of a cerebral blood flow amount obtained in a case where the subjects listened to terrifying music;

FIG. 18D is a graph illustrating time-series data of a cerebral blood flow amount obtained in a case where the subjects listened to white noise;

FIG. 19 illustrates a correlation between a change rate of an average heart rate and a subjective assessment score concerning a level of arousal; and

FIG. 20 is a diagram in which data of cerebral blood flows and heart rates during listening of four types of music were plotted.

### DETAILED DESCRIPTIONS

An embodiment described below illustrates a general or specific example. Numerical values, shapes, materials, constituent elements, the way in which the constituent elements are disposed and connected, steps, the order of steps, and the like illustrated in the embodiment below are examples and do not limit the technique of the present disclosure. Among constituent elements in the embodiment below, constituent elements that are not described in independent claims indicating highest concepts are described as optional constituent elements. Each drawing is a schematic view and is not necessarily strict illustration. Furthermore, in the drawings, substantially identical or similar constituent elements are given identical reference signs. Repeated description may be omitted or simplified.

In the present disclosure, all or a part of any of circuit, unit, device, part or portion, or any of functional blocks in the block diagrams may be, for example, implemented as one or more of electronic circuits including a semiconductor device, a semiconductor integrated circuit (IC), or a large scale integration (LSI). The LSI or IC can be integrated into one chip, or also can be a combination of chips. For example, functional blocks other than a memory may be integrated into one chip. The name used here is LSI or IC, but it may also be called system LSI, very large scale integration (VLSI), or ultra large scale integration (ULSI) depending on the degree of integration. A Field Programmable Gate Array (FPGA) that can be programmed after manufacturing an LSI or a reconfigurable logic device that allows reconfiguration of the connection or setup of circuit cells inside the LSI can be used for the same purpose.

Further, it is also possible that all or a part of the functions or operations of the circuit, unit, device, part or portion are implemented by executing software. In such a case, the software is recorded on one or more non-transitory recording media such as a ROM, an optical disk or a hard disk drive, and when the software is executed by a processor, the software causes the processor together with peripheral devices to execute the functions specified in the software. A system or apparatus may include such one or more non-transitory recording media on which the software is recorded and a processor together with necessary hardware devices such as an interface. Underlying Knowledge Forming Basis of the Present Disclosure

Underlying knowledge forming basis of the present disclosure is described before an embodiment of the present disclosure is described.

There are various methods for measuring brain activity, such as electroencephalography and functional magnetic resonance imaging (fMRI). The electroencephalography is susceptible to noise, and therefore high-accuracy measurement is difficult. The fMRI is most frequently used in research of neuroimaging, but requires a large-size apparatus and forces a subject to be restrained. Therefore, use of the fMRI is restricted to medical use or research use. It is difficult to apply the methods such as the electroencephalography and the fMRI to brain activity measurement in real life.

As compared with these methods, near-infrared spectroscopy (NIRS) has less restraining and high noise resistance characteristics. It is therefore expected that the NIRS is applied to brain activity measurement in real life. The brain activity measurement using the NIRS utilizes high tissue permeability of near-infrared light and wavelength dependence of an absorption coefficient of hemoglobin. As for the high tissue permeability, near-infrared light easily passes through a living body since a near-infrared wavelength region is relatively low in scattering coefficient and light absorption coefficient in a living body and attenuates less in a living body. As for the wavelength dependence, light absorbance of oxyhemoglobin (Oxy-Hb) and light absorbance of deoxyhemoglobin (Deoxy-Hb) in blood are equal at a wavelength close to 803 nm, the light absorbance of the deoxyhemoglobin is higher at a wavelength shorter than 803 nm, and the light absorbance of the oxyhemoglobin is higher at a wavelength longer than 803 nm. Therefore, by utilizing a difference in absorption spectrum between oxyhemoglobin and deoxyhemoglobin, an oxygenated/deoxygenated state of hemoglobin can be approximately measured on the basis of the Beer-Lambert law. According to the Beer-Lambert law, (light absorbance)=(molar absorption coefficient)×(molar concentration of medium)×(medium length: optical path length) is established. On the basis of this relational expression, changes of concentrations of oxyhemoglobin and deoxyhemoglobin from a reference state (e.g., a resting state) can be estimated from a result of measurement of near-infrared light of two wavelengths.

In brain activity measurement using the NIRS, a cerebral blood flow of a frontal lobe of a person is measured. When the measurement is performed, an NIRS device is attached to a forehead. Near-infrared light is emitted from a light emitting part of the NIRS device. The near-infrared light passes through skin, a skull, and a cerebrospinal fluid and then reaches a brain surface. Part of the light is absorbed, and remaining part of the light is scattered and passes through the cerebrospinal fluid, the skull, and the skin again and is then detected by a light receiving part of the NIRS device. Since it is difficult to decide an optical path length of the light detected by the NIRS device, a measured amount is a relative value of a hemoglobin amount. Change amounts of oxyhemoglobin and deoxyhemoglobin in a living body tissue from a reference state can be calculated from an intensity of the detected near-infrared light. When cranial nerve activity becomes active, oxygen consumption in the cranial nerve increases, and a blood flow increases to compensate the increase in oxygen consumption. Oxygen supply by the increased blood flow largely surpasses the increase in oxygen consumption caused by the increase in nerve activity, and therefore oxygen is excessively supplied. Therefore, when brain activity becomes active, Oxy-Hb measured by the NIRS increases. Based on this principle, brain activity can be estimated by using the NIRS.

It is known that amygdala plays a central role in inducing an emotion such as an excited state or a relaxed state. However, not only the amygdala induces an emotion, but also a frontal lobe, which is mainly responsible for thinking, judging, and acting, is involved, and these parts control an emotion by cognition. Therefore, there is a possibility that an emotion can be estimated by measuring a change in blood flow in a surface of the frontal lobe by the NIRS.

However, conventional emotion estimation methods have rooms for improvement in accuracy of estimation of an emotion. Conventional art documents disclosing the conventional emotion estimation methods do not sufficiently disclose data indicating that an emotional state has been estimated with high accuracy. Therefore, it cannot be said that an emotional state can be estimated with high accuracy by the conventional methods.

If an emotional state can be estimated with high accuracy, an improvement in quality of life is expected in various scenes of life such as a home, an interior space of au automobile, an office, a resting room, and an event site. For example, it is expected that a specific emotion such as an excited state or a relaxed state can be induced in a user by controlling a device such as a lighting device or an acoustic device in accordance with an estimated emotion.

However, the conventional emotion estimation methods using cerebral blood flow measurement have the above problem, and therefore cannot estimate an emotional state with high accuracy. In view of this, the inventors of the present invention studied a method for estimating an emotional state such as an excited state or a relaxed state with higher accuracy and arrived at a configuration of the embodiment of the present disclosure.

An outline of the embodiment of the present disclosure is described below.

A method according to an exemplary embodiment of the present disclosure is a method executed by a computer and includes acquiring an amount of change of a cerebral blood flow of a user from a reference time; acquiring an amount of change of a heart rate of the user from a reference time; and outputting a signal indicating that the user is in an excited state in a case where the amount of change of the cerebral blood flow is smaller than a first threshold value and the amount of change of the heart rate is larger than a second threshold value.

The amount of change of the cerebral blood flow from the reference time can be a difference between or a ratio of a cerebral blood flow amount measured at the reference time and a cerebral blood flow amount measured at a measurement time by a cerebral blood flow sensor such as an NIRS device. The cerebral blood flow amount can be, for example, an amount according to a concentration of oxyhemoglobin in cerebral blood. The amount of change of the heart rate from the reference time can be a difference between or a ratio of an average heart rate measured at the reference time and an average heart rate measured at the measurement time by a device that measures a heart rate. The average heart rate is an average of heart rates measured for a certain period from the reference time or the measurement time. The reference time can be, for example, a time at which a specific emotion is not induced such as a resting time. The reference time for measurement of the cerebral blood flow and the reference time for measurement of the heart rate may be identical or may be different. The first threshold value can be, for example, a value smaller than zero (0). The second threshold value is, for example, a value closer to 0 and may be a positive value or a negative value. The first threshold value and the second threshold value may be, for example, set to values that vary from one user to another by calibration. The signal indicating that the user is in an excited state may be, for example, a control signal for displaying information indicating that the user is in the excited state or a degree of excitement on a display. Alternatively, the signal indicating that the user is in an excited state may be a control signal for causing an external device such as a lighting device or an audio output device to execute an operation according to a degree of user's excitement.

According to the above method, whether or not a user is in an excited state can be estimated with high accuracy on the basis of an amount of change of a cerebral blood flow and an amount of change of a heart rate. This, for example, makes it possible to display a degree of user's excitement on a display or control an external device such as a lighting device or an audio output device in accordance with a degree of user's excitement. The above method is based on finding of the inventors of the present invention that when a person is in an excited state, a cerebral blood flow amount such as an oxyhemoglobin amount decreases as compared with that during a resting state. Conventionally, it has been considered that when a person is in an excited state, a cerebral blood flow amount such as an oxyhemoglobin increases as compared with that during a resting state. The inventors of the present invention conducted an experiment for finding out a relationship between an amount of change of a cerebral blood flow and a subject's emotion and thus discovered a fact contrary to the conventional common knowledge, that is, a fact that when a person is in an excited state, a cerebral blood flow amount decreases as compared with that during a resting state. Furthermore, on the basis of an experiment result that a heart rate increases when a person is in an excited state, the inventors of the present invention arrived at the above method. By applying the above method, an excited state of a person can be detected with higher accuracy than conventional arts.

The method may further include outputting a signal indicating that the user is in a relaxed state in a case where the amount of change of the cerebral blood flow is smaller than the first threshold value and the amount of change of the heart rate is smaller than the second threshold value.

The experiment conducted by the inventors of the present invention has revealed that not only in a case where a person is in an excited state, but also in a case where a person is in a relaxed state, a cerebral blood flow amount such as an oxyhemoglobin amount decreases as compared with an amount at the reference time. Furthermore, it has been revealed that when a person is in a relaxed state, a heart rate decreases as compared with that at a reference time, unlike a case where a person is in an excited state. Based on these findings, according to the above method, it is determined that a user is in a relaxed state in a case where the amount of change of the cerebral blood flow is smaller than the first threshold value and the amount of change of the heart rate is smaller than the second threshold value. According to this method, it can be estimated that the user is in a relaxed state with higher accuracy than conventional arts.

As described above, the amount of change of the cerebral blood flow can be an amount of change of oxyhemoglobin in cerebral blood of the user. By applying the above method on the basis of the amount of change of the oxyhemoglobin, it can be estimated with higher accuracy that the user is in an excited state or a relaxed state. The first threshold value can be a value smaller than 0, that is, a negative value. The first threshold value may be set to a value appropriate for the user and may be set to a value equal to or larger than 0. Since the user is not in a neutral emotion at the reference time in some cases, the first threshold value may be set flexibly in accordance with a user's emotional state at the reference time.

The amount of change of the cerebral blood flow may be, for example, acquired while the user is viewing or listening to content including sound and/or an image that induces an excited state or a relaxed state in the user. The reference time may be a time at or before start of the user's viewing or listening of the content. This configuration can be used in a device that provides content inducing an excited state or a relaxed state in a user to the user. According to this configuration, it can be determined to what degree a user is in an excited state or a relaxed state while the user is viewing or listening to content inducing an excited state or a relaxed state in the user. It is therefore possible to perform control such as changing a sound volume of the content or contents of the content in accordance with the degree of the user's excited state or relaxed state. By such control, a degree of user's excitement can be increased, the excitement can be calmed, or the user can be made more relaxed.

As described above, the signal indicating that the user is in the excited state can include at least one of (i) a signal for controlling output of a lighting device or (ii) a signal for controlling output of an audio output device.

According to the above configuration, illuminance of the lighting device can be increased or lowered, a sound volume of sound output from the audio output device can be increased or lowered, or contents of the sound can be changed in accordance with a degree of user's excitement. This can further heighten the degree of user's excitement or can calm the excitement. Note that the signal indicating that the user is in the excited state may include a signal for controlling any device giving some sort of stimulus to a user that is not limited to a lighting device and an audio output device. The stimulus given to the user is not limited to a visual stimulus or an auditory stimulus and may be, for example, a different kind of stimulus such as a haptic stimulus, an olfactory stimulus, or a taste stimulus.

The method may further include outputting at least one of (i) a control signal for increasing illuminance of a lighting device or (ii) a control signal for causing an audio output device to output sound inducing an excited state in the user in a case where at least one of a condition that the amount of change of the cerebral blood flow is larger than the first threshold value or a condition that the amount of change of the heart rate is smaller than the second threshold value is satisfied.

It is estimated that the user is not in an excited state in a case where at least one of a condition that the amount of change of the cerebral blood flow is larger than the first threshold value or a condition that the amount of change of the heart rate is smaller than the second threshold value is satisfied. In such a case, a degree of user's excitement can be heightened by increasing illuminance of the lighting device or outputting sound that induces an excited state in the user.

A method according to another embodiment of the present disclosure is executed by a computer and includes acquiring an amount of change of a cerebral blood flow of a user from a reference time; and outputting a signal indicating that the user is in an excited state or a relaxed state in a case where the amount of change of the cerebral blood flow is smaller than a first threshold value.

According to the above method, it can be estimated that the user is in an excited state or a relaxed state with higher accuracy than conventional arts. According to this method, an amount of change of a heart rate need not necessarily be acquired, unlike the method described earlier. It can be determined whether or not the user is in an excited state or a relaxed state without acquiring an amount of change of a heart rate. That is, it can be determined whether or not the user is in a “pleasant” state corresponding to the right quadrants of the Russell's circumplex model.

The above method may further include acquiring an amount of change of a heart rate of the user from a reference time. The outputting the signal may include outputting a signal indicating that the user is in the excited state in a case where the amount of change of the cerebral blood flow is smaller than the first threshold value and the amount of change of the heart rate is larger than a second threshold value and outputting a signal indicating that the user is in the relaxed state in a case where the amount of change of the cerebral blood flow is smaller than the first threshold value and the amount of change of the heart rate is smaller than the second threshold value. This makes it possible to determine whether the user is in an excited state or a relaxed state with high accuracy.

Each of the above methods can be executed by a computer such as a signal processing circuit of a measurement device. Furthermore, each of the above methods may be executed by a computer such as a server communicably connected to the measurement device over an electric communication line.

A measurement device according to still another embodiment of the present disclosure includes a cerebral blood flow sensor that measures a cerebral blood flow of a user, a heart rate sensor that measures a heart rate of the user, and a signal processing circuit. The signal processing circuit acquires an amount of change of the cerebral blood flow of the user from a reference time, acquires an amount of change of the heart rate of the user from a reference time, and outputs a signal indicating that the user is an excited state in a case where the amount of change of the cerebral blood flow is smaller than a first threshold value and the amount of change of the heart rate is larger than a second threshold value.

A measurement device according to still another embodiment of the present disclosure includes a cerebral blood flow sensor that measures a cerebral blood flow of a user and a signal processing circuit. The signal processing circuit calculates an amount of change of the cerebral blood flow of the user from a reference time on the basis of a signal output from the cerebral blood flow sensor and outputs a signal indicating that the user is in an excited state or a relaxed state in a case where the amount of change of the cerebral blood flow is smaller than a first threshold value.

The present disclosure also includes a computer program causing a computer to execute each of the above methods. A computer program according to an embodiment causes a computer to acquire an amount of change of a cerebral blood flow of a user from a reference time; acquire an amount of change of a heart rate of the user from a reference time; and output a signal indicating that the user is in an excited state in a case where the amount of change of the cerebral blood flow is smaller than a first threshold value and the amount of change of the heart rate is larger than a second threshold value.

A computer program according to another embodiment of the present disclosure causes a computer to acquire an amount of change of a cerebral blood flow of a user from a reference time; and output a signal indicating that the user is in an excited state or a relaxed state in a case where the amount of change of the cerebral blood flow is smaller than a first threshold value.

The computer program can be offered by being stored in a computer-readable non-transitory recording medium. Alternatively, the computer program may be offered over an electric communication line such as the Internet.

A system according to the present disclosure includes a light source that irradiates a head of a user with near-infrared light, a first sensor that detects reflected light that is the near-infrared light reflected by the user, a second sensor that detects a heart rate of the user, an environmental control device that controls an environment around the user, and a circuit, and the circuit generates first information indicative of an amount of change of a concentration of oxyhemoglobin in cerebral blood of the user from a reference time on the basis of the reflected light detected by the first sensor, generates second information indicative of an amount of change of the heart rate from a reference time on the basis of the heart rate detected by the second sensor, and output a control signal for changing the environment around the user to the environmental control device in a case where it is determined that the amount of change of the oxyhemoglobin is smaller than a first threshold value on the basis of the first information and it is determined that the amount of change of the heart rate is larger than a second threshold value on the basis of the second information.

A system according to another embodiment of the present disclosure is configured such that the environmental control device is a lighting device that irradiates the user with illumination light, and the circuit outputs a control signal for lowering at least one of illuminance or a color temperature of the illumination light to the lighting device in a case where it is determined that the amount of change of the oxyhemoglobin is smaller than the first threshold value on the basis of the first information and it is determined that the amount of change of the heart rate is larger than the second threshold value on the basis of the second information.

A system according to another embodiment of the present disclosure is configured such that the environmental control device is a fragrance releasing device that releases fragrance, and the circuit outputs a control signal for causing the fragrance releasing device to release at least one fragrance selected from the group consisting of jasmine, bergamot, rose, lavender, chamomile, cypress, neroli, sandalwood, and cedarwood in a case where it is determined that the amount of change of the oxyhemoglobin is smaller than the first threshold value on the basis of the first information and it is determined that the amount of change of the heart rate is larger than the second threshold value on the basis of the second information.

A system according to another embodiment of the present disclosure is configured such that the environmental control device is an air-conditioning apparatus that performs air-conditioning control on air around the user, and the circuit outputs a control signal for causing the air-conditioning apparatus to supply hot air in a case where it is determined that the amount of change of the oxyhemoglobin is smaller than the first threshold value on the basis of the first information and it is determined that the amount of change of the heart rate is larger than the second threshold value on the basis of the second information.

A system according to another embodiment of the present disclosure is configured such that the environmental control device is an air-conditioning apparatus that performs air-conditioning control on air around the user, and the circuit outputs a control signal for generating a 1/f fluctuation air current around the user to the air-conditioning apparatus in a case where it is determined that the amount of change of the oxyhemoglobin is smaller than the first threshold value on the basis of the first information and it is determined that the amount of change of the heart rate is larger than the second threshold value on the basis of the second information.

A system according to another embodiment of the present disclosure is configured such that the environmental control device is a lighting device that irradiates the user with illumination light, and the circuit outputs a control signal for increasing at least one of illuminance or a color temperature of the illumination light to the lighting device in a case where it is determined that the amount of change of the oxyhemoglobin is smaller than the first threshold value on the basis of the first information and it is determined that the amount of change of the heart rate is smaller than the second threshold value on the basis of the second information.

A system according to another embodiment of the present disclosure is configured such that the environmental control device is a fragrance releasing device that releases fragrance, and the circuit outputs a control signal for causing the fragrance releasing device to release at least one fragrance selected from the group consisting of peppermint, lemon, rosemary, and lemon grass in a case where it is determined that the amount of change of the oxyhemoglobin is smaller than the first threshold value on the basis of the first information and it is determined that the amount of change of the heart rate is smaller than the second threshold value on the basis of the second information.

A system according to another embodiment of the present disclosure is configured such that the environmental control device is an air-conditioning apparatus that performs air-conditioning control on air around the user, and the circuit outputs a control signal for causing the air-conditioning apparatus to supply cool air in a case where it is determined that the amount of change of the oxyhemoglobin is smaller than the first threshold value on the basis of the first information and it is determined that the amount of change of the heart rate is smaller than the second threshold value on the basis of the second information.

A system according to another embodiment of the present disclosure is configured such that the near-infrared light and the reflected light are pulsed light, and the circuit causes the first sensor to start detection of a component of the reflected light during a falling period, which is a period from start of decrease in intensity of the reflected light to end of the decrease.

A more specific embodiment of the present disclosure is described below. Embodiment

### 1. Method for Estimating Emotional State

A method for estimating an emotional state according to an exemplary embodiment of the present disclosure is described.

In the present embodiment, information on an amount of change of a cerebral blood flow from a reference time is acquired on the basis of cerebral blood flow data of a user's frontal lobe acquired by a cerebral blood flow sensor such as an NIRS device. In the present embodiment, an amount of change of oxyhemoglobin in cerebral blood from a reference time is acquired as the amount of change of a cerebral blood flow. Furthermore, a user's electrocardiogram or pulse wave is measured by a heart rate sensor, and an amount of change of an average heart rate from a reference time is acquired on the basis of a measured value. A user's excited state or relaxed state is estimated on the basis of the amount of change of the cerebral blood flow and the amount of change of the average heart rate from the reference time. More specifically, the estimation method according to the present embodiment includes the following steps (A) to (D).

(A) Acquire an amount of change of a user's cerebral blood flow from a reference time.

(B) Acquire an amount of change of a user's average heart rate from a reference time.

(C) Compare the amount of change of the cerebral blood flow with a first threshold value.

(C) Compare the amount of change of the average heart rate with a second threshold value.

(D) Determine that the user is in an excited state in a case where the amount of change of the cerebral blood flow is smaller than the first threshold value and the amount of change of the average heart rate is larger than the second threshold value.

(E) Determine that the user is in a relaxed state in a case where the amount of change of the cerebral blood flow is smaller than the first threshold value and the amount of change of the average heart rate is smaller than the second threshold value.

Note that it is possible that only one of the steps (D) and (E) is executed and the other one of the steps (D) and (E) is not executed.

In the present embodiment, scattering light scattered inside a user's brain is detected by a cerebral blood flow sensor such as an NIRS device at both a reference time and a measurement time. An amount of change of an amount or a concentration of oxyhemoglobin (OxyHb) in cerebral blood from the reference time is acquired as the amount of change of the cerebral blood flow on the basis of a difference between or a ratio of an intensity of scattering light detected at the reference time and an intensity of scattering light detected at the measurement time. The amount of change of the average heart rate is calculated from a measurement value of a heart rate sensor. The average heart rate is an average value of heart rates measured over a certain period (e.g., approximately several seconds to several minutes). In the following description, an amount of change of an average heart rate is simply referred to as an “amount of change of a heart rate”. The amount of change of the cerebral blood flow and the amount of change of the heart rate may be acquired by a single device including both a function of a cerebral blood flow sensor and a function of a heart rate sensor.

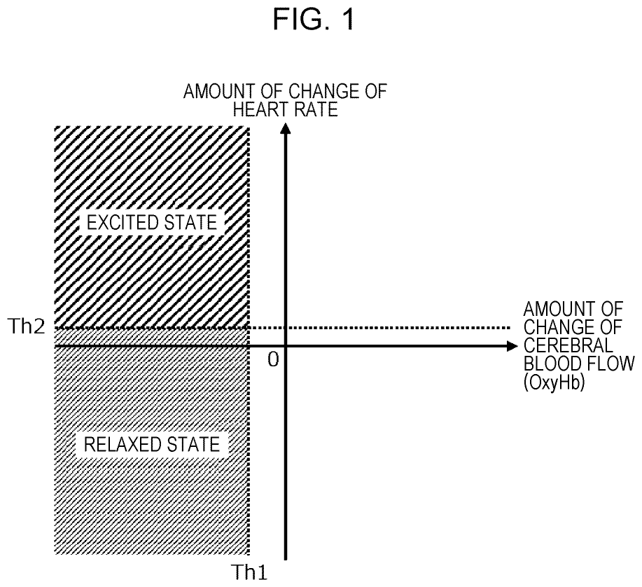

FIG. 1 is a view for explaining a condition on which it is determined that a user is in an excited state or a relaxed state. As illustrated in FIG. 1, it is determined that the user is in an excited state in a case where the amount of change of the cerebral blood flow (in this example, an amount of change of OxyHb) from the reference time is smaller than a first threshold value Th1 and the amount of change of the heart rate is larger than a second threshold value Th2. It is determined that the user is in a relaxed state in a case where the amount of change of the cerebral blood flow from the reference time is smaller than the first threshold value Th1 and the amount of change of the heart rate is smaller than the second threshold value Th2. It is determined that the user is not in an excited state nor a relaxed state in a case where the amount of change of the cerebral blood flow is larger than the first threshold value Th1.

According to such a method, a user's excited state or relaxed state can be estimated with high accuracy on the basis of an amount of change of a cerebral blood flow (in this example, an amount of change of OxyHb) and an amount of change of a heart rate.

In the example of FIG. 1, the first threshold value Th1 is a negative value, and the second threshold value Th2 is a positive value close to 0. This reflects a result of an experiment that will be described later. However, this is merely an example. The first threshold value Th1 and the second threshold value Th2 can be set to appropriate values for each user. For some users, the first threshold value Th1 may be set to a value equal to or larger than 0, and the second threshold value Th may be set to a value equal to or smaller than 0. The first threshold value Th1 and the second threshold value Th2l can be set to optimum values for each user before measurement, for example, by calibration.

The reference time for the amount of change of the cerebral blood flow and the amount of change of the heart rate can be, for example, a time at which the user is in a neutral state, such as a resting state, that is not an excited state nor a relaxed state. An emotion can be estimated while the user is viewing or listening to content such as an image and/or sound that induces an excited state or a relaxed state in the user. In this case, the reference time may be a time of start of output of the content or may be a time earlier than the time of start of output of the content.

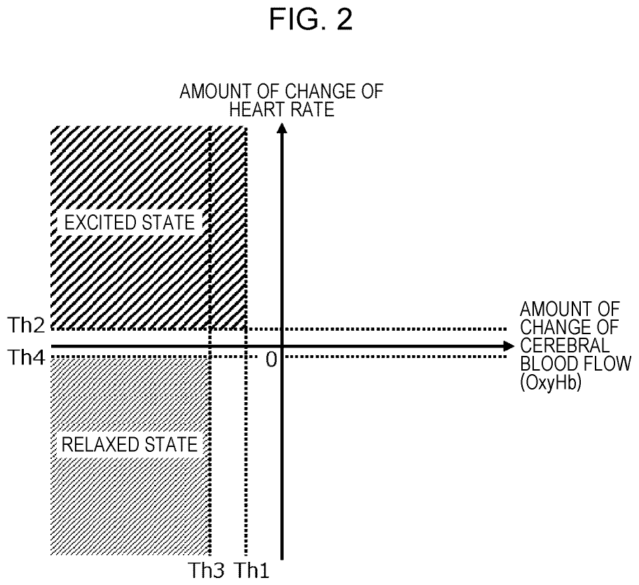

In the example of FIG. 1, the common first threshold value Th1 and second threshold value Th2 are used for both of an excited state and a relaxed state. However, these threshold values for an excited state may be different from those for a relaxed state. For example, as illustrated in FIG. 2, it may be determined that the user is in an excited state in a case where the amount of change of the cerebral blood flow from the reference time is smaller than the first threshold value Th1 and the amount of change of the heart rate is larger than the second threshold value Th2, and it may be determined that the user is in a relaxed state in a case where the amount of change of the cerebral blood flow from the reference time is smaller than a third threshold value Th3 different from the first threshold value Th1 and the amount of change of the heart rate is smaller than a fourth threshold value Th4 different from the second threshold value Th2. In this case, the first threshold value Th1, the second threshold value Th2, the third threshold value Th3, and the fourth threshold value Th4 can be set to appropriate values for each user.

### 2. System for Estimating Emotional State

Next, an example of a system that executes the method for estimating an emotional state according to the present embodiment is described.

### 2.1. Overall Configuration

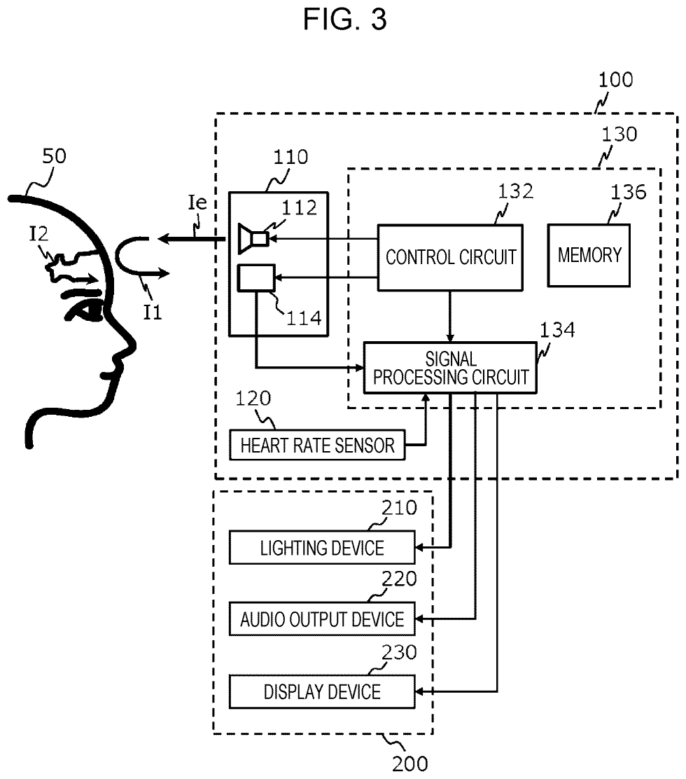

FIG. 3 illustrates an example of a configuration of a system that estimates a user's emotional state. The system illustrated in FIG. 3 includes a measurement device 100 and a stimulation device 200. The measurement device 100 includes a cerebral blood flow sensor 110, a heart rate sensor 120, and a processing device 130. The cerebral blood flow sensor 110 includes a light emitting device 112 and a light receiving device 114. The processing device 130 includes a control circuit 132, a signal processing circuit 134, and a storage medium such as a memory 136. The stimulation device 200 includes a lighting device 210, an audio output device 220, and a display device 230. The stimulation device 200 is a device that gives a stimulus inducing a specific emotion in a user 50. In the example of FIG. 3, the stimulation device 200 gives a visual stimulus to a user by the lighting device 210 and the display device 230 and gives an auditory stimulus to the user by the audio output device 220. The stimulation device 200 may include only any one or two of the lighting device 210, the audio output device 220, and the display device 230. The stimulation device 200 may include not only devices that give a visual stimulus and an auditory stimulus, but also devices that give other kinds of stimuli such as a haptic stimulus and an olfactory stimulus. For example, the stimulation device 200 may include a device such as a massage chair, a vibration generation device, or an odor generation device.

The cerebral blood flow sensor 110 in the example of FIG. 3 is a non-contact-type NIRS device. The light emitting device 112 is disposed so as to emit light toward the forehead of the user 50. Light emitted from the light emitting device 112 and reaching the forehead of the user 50 is separated into a surface reflected component I1 reflected on a surface of the forehead of the user 50 and an internal scattered component I2 scattered inside the forehead. The internal scattered component I2 is a component that is reflected or scattered once or multiple-scattered inside the living body. In a case where light is emitted toward a forehead portion of a person as in the present embodiment, the internal scattered component I2 reaches a portion (e.g., brain) inside the forehead portion that is located 8 mm to 16 mm away from a surface of the forehead portion and returns to the measurement device 100. The surface reflected component I1 includes three components, specifically, a directly reflected component, a diffused reflected component, and a scattered reflected component. The directly reflected component is a reflected component whose incident angle and reflection angle are equal. The diffused reflected component is a component that is reflected by being diffused by irregularities of a surface. The scattered reflected component is a component that is reflected by being scattered by an internal tissue in the vicinity of the surface. The scattered reflected component is a component that is reflected by being scattered inside a surface layer of skin. The surface reflected component I1 can include these three components. The surface reflected component I1 and the internal scattered component I2 change their traveling directions due to reflection or scattering, and a part thereof reaches the light receiving device 114. The surface reflected component I1 includes information on a surface of a portion to be measured, for example, information on a blood flow of a face and scalp. The internal scattered component I2 includes information on a user's inside, for example, information on a cerebral blood flow.

In the present embodiment, the internal scattered component I2 of reflected light reflected back from the head of the user 50 is detected. An intensity of the internal scattered component I2 fluctuates reflecting brain activity of the user 50. It is therefore possible to estimate a state of brain activity of the user 50 by analyzing a temporal change of the internal scattered component I2.

The light receiving device 114 is a device that includes one or more photodetectors such as an image sensor. The light receiving device 114 detects the internal scattered component I2 of a reflected light pulse emitted from the light emitting device 112 and then reflected back from the forehead of the user 50 and outputs an electric signal according to an intensity of the component.

The heart rate sensor 120 can include an electrode pad attached to one or more portions such as a wrist, an ankle, or a chest of the user 50. The heart rate sensor 120 measures a heart rate of the user 50 by measuring a weak electric pulse generated from the heart. Note that the heart rate sensor 120 is not limited to one including an electrode pad, and may have a configuration that irradiates a skin surface with near-infrared light, detects reflected light by a photodetector such as a photodiode, and measures a pulse wave by utilizing a near-infrared light absorbing property of hemoglobin in an arterial vessel. In this case, the cerebral blood flow sensor 110 and the heart rate sensor 120 may be a same device. The heart rate sensor 120 may include a camera and a processor that estimates a heart rate by analyzing an image acquired by the camera. Hemoglobin in blood has a property of absorbing, for example, green light. By analyzing a fluctuation in intensity of light reflected by a skin surface caused by contraction and expansion of a blood vessel by utilizing this property, a pulse wave signal can be extracted from an image obtained by imaging a skin surface of a face or the like by the camera.

The control circuit 132 is a circuit that controls the light emitting device 112 and the light receiving device 114. The control circuit 132 causes the light receiving device 114 to perform a detection operation at a timing when at least a part of a rear end component of a reflected light pulse reaches the light receiving device 114. The rear end component of the reflected light pulse is a component from start of decrease in intensity of the reflected light pulse that has reached a light receiving surface of the light receiving device 114 to end of the decrease. By detecting at least a part of the rear end component of the reflected light pulse, the internal scattered component I2 can be detected.

The signal processing circuit 134 generates a cerebral blood flow signal indicative of a state of a cerebral blood flow of the user 50 on the basis of an electric signal output from the light receiving device 114 of the cerebral blood flow sensor 110. The cerebral blood flow signal can be, for example, a signal indicative of a temporal change in concentration of oxyhemoglobin in blood in the brain of the user 50. The signal processing circuit 134 can generate brain activity data indicative of a state of brain activity of the user and/or a control signal for controlling the stimulation device 200 on the basis of the cerebral blood flow signal and the signal indicative of the heart rate output from the heart rate sensor 120. The brain activity data can be data indicative of an emotion of the user 50, such as an excited state or a relaxed state, that is generated on the basis of the cerebral blood flow signal.

The lighting device 210 is a device that is disposed around the user 50 and illuminates the user 50. The lighting device 210 can change a visual stimulus given to the user 50 by changing luminance and/or a wavelength of light with which the user 50 is irradiated. For example, in a case where an excited state is induced in the user 50, the lighting device 210 may irradiate the user 50 with bright light or light of a color of a high color temperature. Conversely, in a case where a relaxed state is induced in the user 50, the lighting device 210 may irradiate the user 50 with dark light or light of a color of a low color temperature.

The audio output device 220 includes a device such as a speaker or a sound reproducer. The audio output device 220 can change an auditory stimulus given to the user 50 by changing a sound volume and/or contents of output sound. For example, in a case where an excited state is induced in the user 50, the audio output device 220 may increase an output sound volume or reproduce up-tempo music. Conversely, in a case where a relaxed state is induced in the user 50, the audio output device 220 may lower the output sound volume or reproduce slow-tempo music.

The display device 230 can be any display such as a liquid crystal display or an OLED display. The display device 230 displays an image based on brain activity data generated by the signal processing circuit 134. The display device 230 may display, for example, an image indicative of a degree of an excited state or a degree of a relaxed state of the user 50. The display device 230 may display image content that gives a visual stimulus to the user 50. The display device 230 can change a visual stimulus given to the user 50 by changing the displayed content.

### 2-2. Cerebral Blood Flow Sensor

Next, a configuration and operation of the cerebral blood flow sensor 110 are described in more detail.

The light emitting device 112 repeatedly emits a light pulse plural times at predetermined time intervals or at predetermined timings in accordance with an instruction from the control circuit 132. The light pulse emitted from the light emitting device 112 can be, for example, a rectangular wave whose falling period has a length close to zero. In the present specification, the “falling period” refers to a period from start of decrease in intensity of a light pulse to end of the decrease. Typically, light incident on the head of the user 50 propagates through the head while passing various routes and is emitted from a surface of the head at different timings. Accordingly, a rear end of the internal scattered component I2 of the light pulse has an expanse. In a case where the portion to be measured is a forehead, the expanse of the rear end of the internal scattered component I2 is approximately 4 ns. In consideration of this, the length of the falling period of the light pulse can be, for example, set equal to or less than a half of this value, that is, equal to or less than 2 ns. The falling period may be equal to or less than 1 ns, which is a half of 2 ns. A rising period of the light pulse emitted from the light emitting device 112 can have any length. In the present specification, the “rising period” refers to a period from start of increase in intensity of the light pulse to end of the increase. In detection of the internal scattered component I2 according to the present embodiment, the falling part of the light pulse is used, and the rising part of the light pulse is not used. The rising part of the light pulse is used for detection of the surface reflected component I1.

The light emitting device 112 includes one or more light sources. The light source can include, for example, a laser element such as a laser diode (LD). Light emitted from the laser element can be adjusted to have steep time response characteristics such that a falling part of a light pulse is substantially orthogonal to a time axis. The light emitting device 112 can include a drive circuit that controls a drive current of the LD. The drive circuit can include, for example, an enhancement mode power transistor such as a field-effect transistor (GaNFET) including a gallium nitride (GaN) semiconductor. By using such a drive circuit, falling of the light pulse output from the LD can be made steep.

Light emitted from the light emitting device 112 can have, for example, any wavelength included in a wavelength range equal to or longer than 650 nm and equal to or shorter than 950 nm. This wavelength range is included in a wavelength range from red to near-infrared rays. This wavelength range is called a “biological window” and has such a property that light is relatively hard to be absorbed by moisture in a living body and skin. In a case where a living body is used as a detection target, detection sensitivity can be increased by using light in this wavelength range. Note that in the present specification, a term “light” is used not only for visible light, but also for an infrared ray. In a case where a change in cerebral blood flow of a person is detected as in the present embodiment, used light is considered to be mainly absorbed by oxyhemoglobin and deoxyhemoglobin. The oxyhemoglobin and the deoxyhemoglobin are different in wavelength dependence of light absorption. In general, when a blood flow changes in accordance with brain activity, a concentration of the oxyhemoglobin and a concentration of the deoxyhemoglobin change. A degree of absorption of light also changes in accordance with this change. Accordingly, when the blood flow changes, an amount of detected light also changes temporally. By detecting the change in amount of light, a state of brain activity can be estimated. The light emitting device 112 may emit light of a single wavelength included in the wavelength range or may emit light of two or more wavelengths included in the wavelength range. The light of the wavelengths may be emitted from light sources.

In general, absorption characteristics and scattering characteristics of a living body tissue vary depending on a wavelength. Therefore, more detailed component analysis of a target to be measured can be conducted by detecting wavelength dependence of an optical signal based on the internal scattered component I2. For example, in a living body tissue, in a case where the wavelength is equal to or longer than 650 nm and is shorter than 805 nm, a coefficient of light absorption by deoxyhemoglobin is larger than a coefficient of light absorption by oxyhemoglobin. On the other hand, in a case where the wavelength is longer than 805 nm and is equal to or shorter than 950 nm, the coefficient of light absorption by oxyhemoglobin is larger than the coefficient of light absorption by deoxyhemoglobin.

Therefore, the light emitting device 112 may be configured to emit light of a wavelength equal to or longer than 650 nm and shorter than 805 nm (e.g., approximately 750 nm) and light of a wavelength longer than 805 nm and equal to or shorter than 950 nm (e.g., approximately 850 nm). In this case, a light intensity of the internal scattered component I2 generated, for example, by light of a wavelength of approximately 750 nm and a light intensity of the internal scattered component I2 generated, for example, by light of a wavelength of approximately 850 nm can be measured. The light emitting device 112 may include a light source that emits light of a wavelength equal to or longer than 650 nm and shorter than 805 nm and a light source that emits light of a wavelength longer than 805 nm and equal to or shorter than 950 nm. The signal processing circuit 134 can find change amounts of concentrations of oxyhemoglobin and deoxyhemoglobin in the blood from initial values by solving predetermined simultaneous equations on the basis of signal values of the light intensities input for each pixel.

The measurement device 100 according to the present embodiment can measure a cerebral blood flow amount of the user 50 in a non-contact manner. For this purpose, the light emitting device 112 designed in consideration of influence on a retina can be used. For example, the light emitting device 112 that satisfies Class 1 of the laser safety standards adopted in various countries can be used. In a case where Class 1 is satisfied, the user 50 is irradiated with light having such low illuminance that an accessible emission limit (AEL) is smaller than 1 mW. Note that the light emitting device 112 itself need not satisfy Class 1. For example, Class 1 of the laser safety standards may be satisfied by disposing a diffusion plate or an ND filter in front of the light emitting device 112 and thereby diffusing or attenuating light.

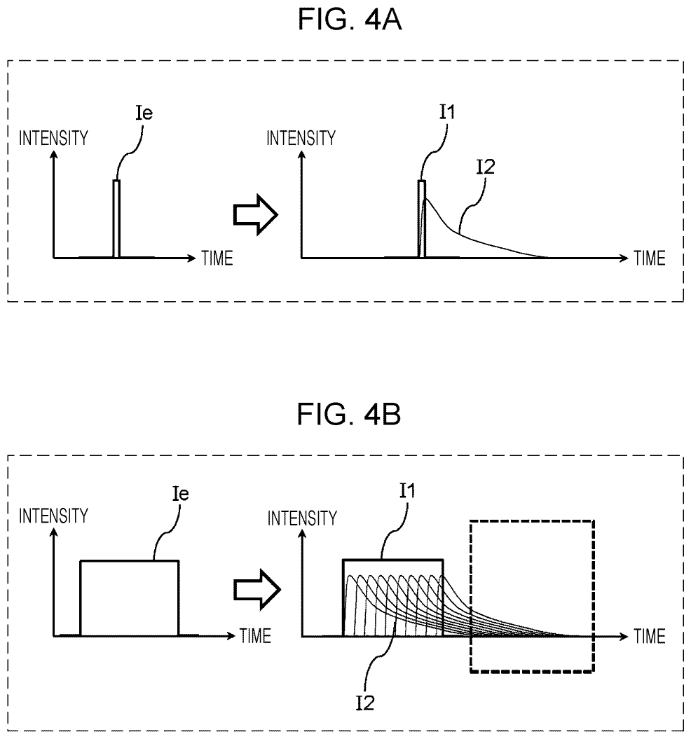

FIGS. 4A and 4B illustrate examples of temporal changes of the intensity of the emitted light pulse Ie and the intensities of the surface reflected component I1 and the internal scattered component I2 in the reflected light pulse. FIG. 4A illustrates an example of waveforms obtained in a case where the emitted light pulse Ie has an impulse waveform. FIG. 4B illustrates an example of waveforms obtained in a case where the emitted light pulse Ie has a rectangular waveform. Although the internal scattered component I2 is actually weak, the intensity of the internal scattered component I2 is emphasized in FIGS. 4A and 4B.

As illustrated in FIG. 4A, in a case where the emitted light pulse Ie has an impulse waveform, the surface reflected component I1 has an impulse waveform similar to the light pulse Ie, and the internal scattered component I2 has an impulse response waveform delayed relative to the surface reflected component I1. This is because the internal scattered component I2 corresponds to a combination of light beams that have passed various routes inside the skin.

As illustrated in FIG. 4B, in a case where the light pulse Ie has a rectangular waveform, the surface reflected component I1 has a rectangular waveform similar to the light pulse Ie, and the internal scattered component I2 has a waveform in which a large number of impulse response waveforms are superimposed. The inventors of the present invention confirmed that the light amount of the internal scattered component I2 detected by the light receiving device 114 can be amplified by superimposition of a large number of impulse response waveforms as compared with a case where the light pulse Ie has an impulse waveform. The internal scattered component I2 can be effectively detected by starting opening of an electronic shutter at or after a timing of start of falling of the reflected light pulse. The broken-line frame on the right side of FIG. 4B illustrates an example of a shutter opening period for which the electronic shutter of the light receiving device 114 is opened. This shutter opening period is also referred to as an “exposure period”. The internal scattered component I2 can be effectively detected by starting exposure at or after a timing of start of falling of the surface reflected component I1 reaching the light receiving device 114.

The light emitting device 112 can include, for example, a light-emitting element using a general-purpose semiconductor laser. The light emitting device 112 can be, for example, controlled to emit a light pulse having a pulse width of 3 ns or more to obtain a stable waveform by using a general-purpose semiconductor laser. Alternatively, the light emitting device 112 may emit a light pulse having a pulse width of 5 ns or more or a pulse width of 10 ns or more to further stabilize the waveform. On the other hand, setting the pulse width too large increases a light flow to a charge accumulation unit 124 in a shutter OFF state, that is, increases parasitic light sensitivity (PLS), leading to a risk of a measurement error. In view of this, the light emitting device 112 can be, for example, controlled to generate a light pulse having a pulse width of 50 ns or less. Alternatively, the light emitting device 112 may emit a light pulse having a pulse width of 30 ns or less or a pulse width of 20 ns or less.

Next, an example of a configuration of the light receiving device 114 is described in more detail.

The light receiving device 114 can be, for example, any image sensor such as a CCD image sensor or a CMOS image sensor. The light receiving device 114 includes photodetection cells that are two-dimensionally disposed on a light receiving surface. Each of the photodetection cells can include, for example, a photoelectric conversion element such as a photodiode and one or more charge accumulation units. The photoelectric conversion element generates a signal charge according to an amount of received light by photoelectric conversion. The charge accumulation unit accumulates the signal charge generated by the photoelectric conversion element. The light receiving device 114 can acquire two-dimensional information of a user at one time. In the present specification, the photodetection cells are sometimes referred to as “pixels”.

The light receiving device 114 according to the present embodiment includes an electronic shutter. The electronic shutter is a circuit that controls a timing of exposure. The electronic shutter controls a period of one signal accumulation for which received light is converted into an effective electric signal and is accumulated and a period for which the signal accumulation is stopped. The signal accumulation period is referred to as an “exposure period”. A period from end of one exposure period to start of a next exposure period is referred to as a “non-exposure period”. Hereinafter, a state where exposure is being performed is sometimes referred to as “OPEN”, and a state where the exposure is stopped is sometimes referred to as “CLOSED”.

The light receiving device 114 can adjust the exposure period and the non-exposure period within a subnanosecond range, for example, a range from 30 ps to 1 ns by the electronic shutter. The exposure period and the non-exposure period can be, for example, set to a value equal to or larger than 1 ns and equal to or smaller than 30 ns.

In a case where information such as a cerebral blood flow is detected by irradiating a forehead of a person with light, a rate of attenuation of light inside the living body is very large. For example, emitted light can attenuate to approximately one-millionth of incident light. Accordingly, a light amount sufficient to detect an internal scattered component cannot be sometimes obtained by irradiation of one pulse. Especially in a case of irradiation of Class 1 of the laser safety standards, a light amount is weak. In this case, the control circuit 132 causes the light emitting device 112 to emit a light pulse plural times, and the photodetection cells of the light receiving device 114 are exposed to light plural times in synchronization with this. By thus integrating signals plural times, sensitivity can be improved.

The following describes an example in which each pixel of the light receiving device 114 includes a photoelectric conversion element such as a photodiode and charge accumulation units. In the following example, the charge accumulation units in each pixel include a charge accumulation unit that accumulates a signal charge generated by a surface reflected component of a light pulse and a charge accumulation unit that accumulates a signal charge generated by an internal scattered component of the light pulse. The control circuit 132 causes the light receiving device 114 to detect a component before start of falling in a reflected light pulse reflected back from a forehead of a user and thereby detect a surface reflected component. The control circuit 132 causes the light receiving device 114 to detect a component after start of falling in a light pulse reflected back from a portion to be measured of the user and thereby detect an internal scattered component. Note that detection of the surface reflected component is not essential and may be omitted depending on an intended purpose.

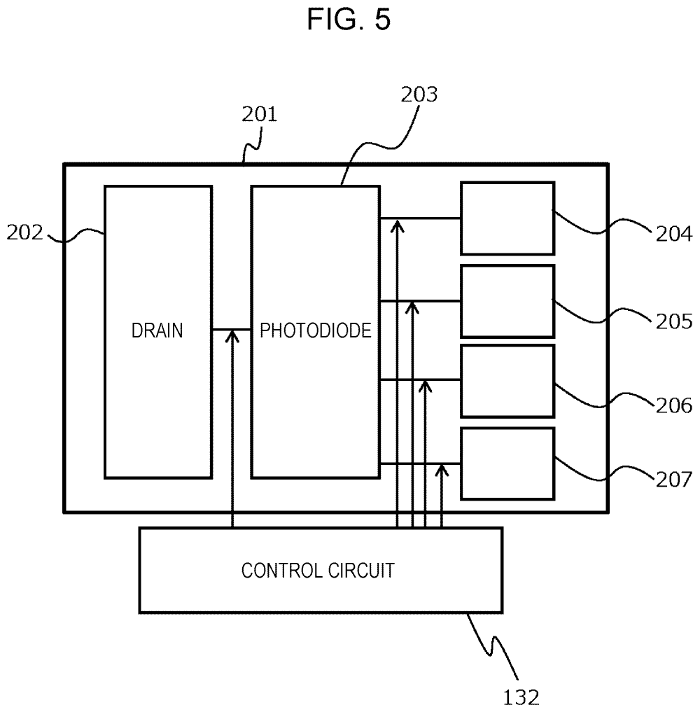

FIG. 5 illustrates an example of an outline configuration of one pixel 201 of the light receiving device 114. Note that FIG. 5 schematically illustrates the configuration of one pixel 201, and an actual structure is not necessarily reflected in FIG. 5. The pixel 201 in this example includes a photodiode 203 that performs photoelectric conversion, a first floating diffusion (FD) layer 204, a second floating diffusion layer 205, a third floating diffusion layer 206, and a fourth floating diffusion layer 207, which are charge accumulation units, and a drain 202 to which a signal charge is discharged. In the example illustrated in FIG. 5, the light emitting device 112 emits light pulses of two kinds of wavelengths.

A photon entering each pixel due to one emission of a light pulse is converted into a signal electron, which is a signal charge, by the photodiode 203. The signal electron is discharged to the drain 202 or is sorted into any one of the first to fourth floating diffusion layers 204 to 207 in accordance with a control signal input from the control circuit 132 to the light receiving device 114.

Emission of a light pulse from the light emitting device 112, accumulation of a signal charge in any of the first floating diffusion layer 204, the second floating diffusion layer 205, the third floating diffusion layer 206, and the fourth floating diffusion layer 207, and discharge of a signal charge to the drain 202 are repeatedly performed in this order. This operation is repeated at a high rate, and can be, for example, repeated several tens of thousands to several hundreds of millions of times within a period of one frame of a moving image. The period of one frame can be, for example, approximately 1/30 seconds. The pixel 201 finally generate and output, for each frame, four image signals based on the signal charges accumulated in the first to fourth floating diffusion layers 204 to 207.

The control circuit 132 according to the present embodiment causes the light emitting device 112 to emit a first light pulse having a first wavelength λ1 and a second light pulse having a second wavelength λ2. An internal state of a portion to be measured can be analyzed by selecting two wavelengths that are different in rate of absorption in an internal tissue of the portion to be measured as the wavelengths λ1 and λ2. For example, a wavelength equal to or longer than 650 nm and shorter than 805 nm can be selected as the wavelength λ1, and a wavelength longer than 805 nm and equal to or shorter than 950 nm can be selected as the wavelength λ2. This makes it possible to efficiently detect a change in oxyhemoglobin concentration and a change in deoxyhemoglobin concentration in the blood of the user 50, as described later.

The control circuit 132 performs, for example, the following operation. The control circuit 132 causes the light emitting device 112 to emit a light pulse of the wavelength λ1, and causes the first floating diffusion layer 204 to accumulate a signal charge during a period where an internal scattered component of the light pulse is incident on the photodiode 203. The control circuit 132 causes the light emitting device 112 to emit the light pulse of the wavelength λ, and causes the second floating diffusion layer 205 to accumulate a signal charge during a period where a surface reflected component of the light pulse is incident on the photodiode 203. Furthermore, the control circuit 132 causes the light emitting device 112 to emit a light pulse of the wavelength λ2, and causes the third floating diffusion layer 206 to accumulate a signal charge during a period where an internal scattered component of the light pulse is incident on the photodiode 203. The control circuit 132 causes the light emitting device 112 to emit a light pulse of the wavelength λ2, and causes the fourth floating diffusion layer 207 to accumulate a signal charge during a period where a surface reflected component of the light pulse is incident on the photodiode 203. The above operation can be repeated plural times. By such an operation, an image showing a two-dimensional distribution of the surface reflected component and an image showing a two-dimensional distribution of the internal scattered component can be acquired for both of the wavelength λ1 and the wavelength λ2.

To estimate light amounts of disturbance light and environment light, a period where a signal charge is accumulated in another floating diffusion layer in a state where the light emitting device 112 is off may be provided. A signal excluding disturbance light and environment light components can be obtained by subtracting a signal charge amount of the other floating diffusion layer from signal charge amounts of the first to fourth first floating diffusion layers 204 to 207.

Note that although the number of charge accumulation units of each pixel is four in the present embodiment, the number of charge accumulation units of each pixel may be set to any number of 1 or more depending on a purpose. For example, in a case where a surface reflected component and an internal scattered component are detected by using one kind of wavelength, the number of charge accumulation units may be two. In a case where one kind of wavelength is used and a surface reflected component is not detected, the number of charge accumulation units of each pixel may be one. In a case where only an internal scattered component is detected by using two kinds of wavelengths, the number of charge accumulation units of each pixel may be two. Even in a case where two or more kinds of wavelengths are used, the number of charge accumulation units may be one, as long as imaging using one wavelength and imaging using another wavelength are performed in different frames. Similarly, even in a case where both of a surface reflected component and an internal scattered component are detected, the number of charge accumulation units may be one, as long as the surface reflected component and the internal scattered component are detected in different frames.

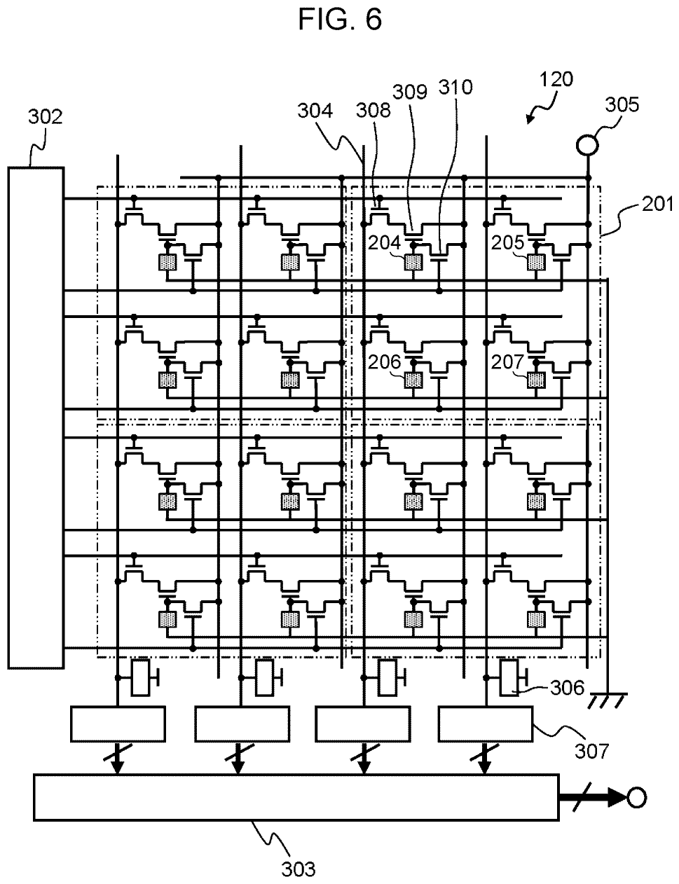

Next, an example of the configuration of the light receiving device 114 is described in more detail with reference to FIG. 6.

FIG. 6 is a diagram illustrating an example of the configuration of the light receiving device 114. In FIG. 6, a region surrounded by the line with alternate long and two short dashes corresponds to a single pixel 201. The pixel 201 includes a single photodiode. Although only four pixels arranged in two rows and two columns are illustrated in FIG. 6, a larger number of pixels can be disposed actually. The pixel 201 includes the first to fourth floating diffusion layers 204 to 207. Signals accumulated in the first to fourth floating diffusion layers 204 to 207 are handled as if these signals are signals of four pixels of a general CMOS image sensor, and are output from the light receiving device 114.

Each pixel 201 has four signal detection circuits. Each signal detection circuit includes a source follower transistor 309, a row selection transistor 308, and a reset transistor 310. In this example, the reset transistor 310 corresponds to the drain 202 illustrated in FIG. 5, and a pulse input to a gate of the reset transistor 310 corresponds to the drain discharge pulse. Each transistor is, for example, a field-effect transistor provided on a semiconductor substrate but is not limited to this. As illustrated in FIG. 6, one of an input terminal and an output terminal of the source follower transistor 309 is connected to one of an input terminal and an output terminal of the row selection transistor 308. The one of the input terminal and the output terminal of the source follower transistor 309 is typically a source. The one of the input terminal and the output terminal of the row selection transistor 308 is typically a drain. A gate of the source follower transistor 309, which is a control terminal, is connected to the photodiode 203. A signal charge, which is a hole or an electron, generated by the photodiode 203 is accumulated in a floating diffusion layer, which is a charge accumulation unit, provided between the photodiode 203 and the source follower transistor 309.

The first to fourth floating diffusion layers 204 to 207 are connected to the photodiode 203 (not illustrated in FIG. 6). One or more switches can be provided between the photodiode 203 and each of the first to fourth floating diffusion layers 204 to 207. The switch switches a conduction state between the photodiode 203 and each of the first to fourth floating diffusion layers 204 to 207 in accordance with a signal accumulation pulse from the control circuit 132. In this way, start and stop of accumulation of a signal charge in each of the first to fourth floating diffusion layers 204 to 207 are controlled. The electronic shutter according to the present embodiment has a mechanism for such exposure control.

Signal charges accumulated in the first to fourth floating diffusion layers 204 to 207 are read out when a gate of the row selection transistor 308 is turned on by a row selection circuit 302. At this time, a current flowing from a source follower power source 305 to the source follower transistor 309 and a source follower load 306 is amplified in accordance with the signal charges of the first to fourth floating diffusion layers 204 to 207. An analog signal based on this current read out from a vertical signal line 304 is converted into a digital signal data by an analog-digital (AD) conversion circuit 307 connected to each column. This digital signal data is read out for each column by a column selection circuit 303 and is output from the light receiving device 114. The row selection circuit 302 and the column selection circuit 303 perform readout in one row and then perform readout in a next row. Thereafter, similarly, information on signal charges of floating diffusion layers in all rows is read out. The control circuit 132 turns the gate of the reset transistor 310 on after all signal charges are read out, and thereby resets all floating diffusion layers. This completes imaging of one frame. Thereafter, similarly, high-rate imaging of a frame is repeated, and thereby imaging of a series of frames by the light receiving device 114 is completed.

Although an example in which a CMOS-type light receiving device 114 is used has been described in the present embodiment, the light receiving device 114 may be another kind of imaging element. For example, the light receiving device 114 may be a CCD type, may be a single photon counting type element, or may be an amplification type image sensor such as an EMCCD or an ICCD. Furthermore, sensors each including a single photoelectric conversion element may be used instead of the light receiving device 114 having photodetection cells that are two-dimensionally arranged. Even in a case where single-pixel sensors are two-dimensionally arranged, two-dimensional data of a portion to be measured can be generated.

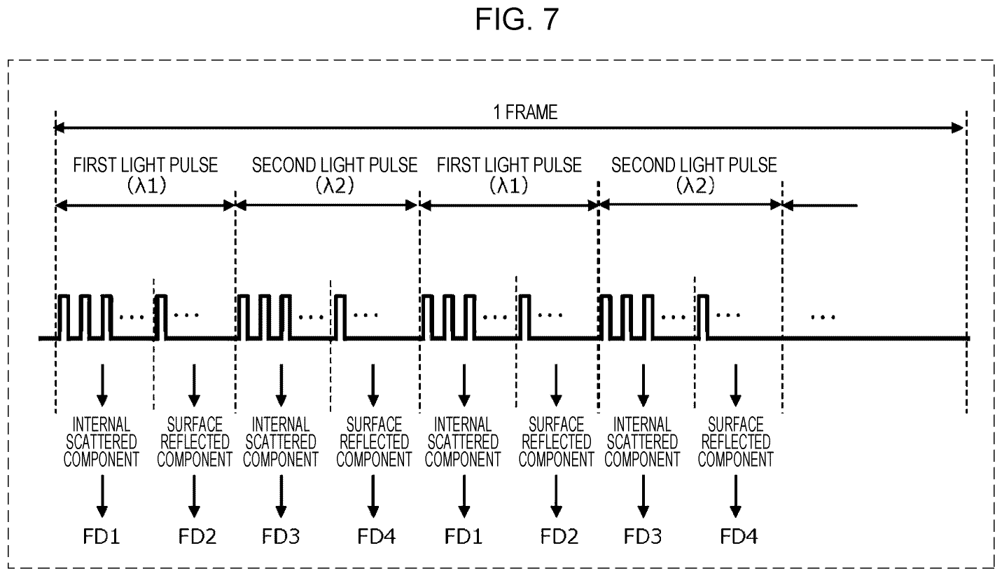

FIG. 7 schematically illustrates an example of an operation performed in one frame. In the example illustrated in FIG. 7, a period for which the first light pulse of the wavelength λ1 is repeatedly emitted and a period for which the second light pulse of the wavelength λ2 is repeatedly emitted alternate within a single frame. The period for which the first light pulse is repeatedly emitted and the period for which the second light pulse is repeatedly emitted each include a period for which a signal charge of an internal scattered component is accumulated and a period for which a signal charge of a surface reflected component is accumulated. The internal scattered component of the light pulse of the wavelength λ1 is accumulated in the first floating diffusion layer 204 (FD1). The surface reflected component of the light pulse of the wavelength λ1 is accumulated in the second floating diffusion layer 205 (FD2). The internal scattered component of the light pulse of the wavelength λ2 is accumulated in the third floating diffusion layer 206 (FD3). The surface reflected component of the light pulse of the wavelength λ2 is accumulated in the fourth floating diffusion layer 207 (FD4). In this example, the control circuit 132 repeats the following operations (i) to (iv) plural times within a one-frame period.

(i) An operation of causing the light emitting device 112 to emit the light pulse of the wavelength λ1 and causing the first floating diffusion layer 204 of each pixel to accumulate the internal scattered component of the light pulse of the wavelength λ1 is repeated a predetermined number of times.

(ii) An operation of causing the light emitting device 112 to emit the light pulse of the wavelength λ1 and causing the second floating diffusion layer 205 of each pixel to accumulate the surface reflected component of the light pulse of the wavelength λ1 is repeated plural times.

(iii) An operation of causing the light emitting device 112 to emit the light pulse of the wavelength λ2 and causing the third floating diffusion layer 206 of each pixel to accumulate the internal scattered component of the light pulse of the wavelength λ2 is repeated a predetermined number of times.

(iv) An operation of causing the light emitting device 112 to emit the light pulse of the wavelength λ2 and causing the fourth floating diffusion layer 207 of each pixel to accumulate the surface reflected component of the light pulse of the wavelength λ2 is repeated plural times.

By such operations, a temporal difference between timings of acquisition of detection signals using two kinds of wavelengths can be reduced, and imaging using the first light pulse and imaging using the second light pulse can be performed almost simultaneously.

In the present embodiment, the light receiving device 114 detects a surface reflected component and an internal scattered component for each of the first light pulse and the second light pulse and generate an image signal indicative of an intensity distribution of each component. A cerebral blood flow signal of the user 50 can be generated for each pixel or each pixel group on the basis of an image signal indicative of an intensity distribution of the internal scattered component of each of the first light pulse and the second light pulse. On the other hand, an image signal indicative of an intensity distribution of the surface reflected component of each of the first light pulse and the second light pulse indicates a face image of the user 50. On the basis of a temporal change of the face image signal, the signal processing circuit 134 can decide a region of the forehead of the user 50 and generate brain activity data by using a detection signal in the decided region.

Note that the light emitting device 112 may emit light of one kind of wavelength. Even in this case, an approximate state of brain activity can be estimated.

The cerebral blood flow sensor 110 may include an imaging optical system that forms a two-dimensional image of the user 50 on the light receiving surface of the light receiving device 114. An optical axis of the imaging optical system is substantially orthogonal to the light receiving surface of the light receiving device 114. The imaging optical system may include a zoom lens. When a position of the zoom lens changes, a magnification of the two-dimensional image of the user 50 changes, and resolution of the two-dimensional image on the light receiving device 114 changes. Therefore, a desired measurement region can be enlarged and observed in detail even in a case where a distance to the user 50 is long.

The cerebral blood flow sensor 110 may include, between the user 50 and the light receiving device 114, a bandpass filter that allows light of a wavelength band emitted from the light emitting device 112 or light in the vicinity of the wavelength band to pass therethrough. This can reduce influence of a disturbance component such as environment light. The bandpass filter can be, for example, a multi-layer filter or an absorption filter. The bandpass filter may have, for example, a bandwidth range of approximately 20 nm to 100 nm in consideration of a band shift resulting from a change in temperature of the light emitting device 112 and oblique incidence on the filter.

The cerebral blood flow sensor 110 may include a polarization plate between the light emitting device 112 and the user 50 and between the light receiving device 114 and the user 50. In this case, a polarization direction of the polarization plate disposed on the light emitting device 112 side and a polarization direction of the polarization plate disposed on the light receiving device 114 side can have a relationship of crossed Nicols. This can prevent a specular reflection component of a surface reflected component of the user 50, that is, a component whose incident angle and reflection angle are identical from reaching the light receiving device 114. That is, it is possible to reduce a light amount of the surface reflected component reaching the light receiving device 114.

### 2-3. Processing Device

Next, a configuration and operation of the processing device 130 are described in more detail.

The control circuit 132 controls the above operations of the light emitting device 112 and the light receiving device 114. Specifically, the control circuit 132 adjusts a time difference between an emission timing of a light pulse of the light emitting device 112 and a shutter timing of the light receiving device 114. The “emission timing” of the light emitting device 112 refers to a timing of start of rising of a light pulse emitted from the light emitting device 112. The “shutter timing” refers to a timing of start of exposure.

The control circuit 132 can be, for example, a processor such as a central processing unit (CPU) or an integrated circuit such as a microcontroller including a processor. The control circuit 132 adjusts the emission timing and the shutter timing, for example, by execution of a computer program recorded in the memory 136 by the processor.

The signal processing circuit 134 is a circuit that processes a signal output from the light receiving device 114. The signal processing circuit 134 performs arithmetic processing such as processing for estimating an emotion of the user 50. The signal processing circuit 134 can be, for example, realized by a digital signal processor (DSP), a programmable logic device (PLD) such as a field programmable gate array (FPGA), a central processing unit (CPU), or a graphics processing unit (GPU). The signal processing circuit 134 performs processing that will be described later by execution of a computer program stored in the memory 136 by a processor.

The memory 136 is a recording medium such as a ROM or a RAM in which computer programs executed by the control circuit 132 and the signal processing circuit 134 and various kinds of data generated by the control circuit 132 and the signal processing circuit 134 are recorded.

The control circuit 132 and the signal processing circuit 134 may be a single unified circuit or may be separate individual circuits. The control circuit 132 and the signal processing circuit 134 may each include circuits. At least one function of the signal processing circuit 134 may be a constituent element of an external device such as a server computer provided separately from the light emitting device 112 and the light receiving device 114. In this case, the external device transmits and receives data to and from the measurement device including the light emitting device 112, the light receiving device 114, and the control circuit 132 through wireless communication or wired communication.

The signal processing circuit 134 can generate a cerebral blood flow signal reflecting the internal scattered component I2 on the basis of a signal output from the light receiving device 114. The signal processing circuit 134 can generate data indicative of a temporal change of a concentration of oxyhemoglobin in blood inside the forehead of the user 50 on the basis of a signal of each pixel output from the light receiving device 114. The signal processing circuit 134 can generate brain activity data indicative of an emotion (e.g., an excited state or a relaxed state) of the user 50 on the basis of the data.

The signal processing circuit 134 may estimate an offset component resulting from disturbance light included in a signal output from the light receiving device 114 and remove the offset component. The offset component is a signal component resulting from disturbance light such as solar light or fluorescent light. The offset component resulting from environment light or disturbance light is estimated by causing the light receiving device 114 to detect a signal in a state where no light is emitted by turning driving of the light emitting device 112 off.

### 2-4. Example of Operation of Cerebral Blood Flow Sensor

Next, operation of the measurement device 100 is described.

The measurement device 100 according to the present embodiment can detect the surface reflected component I1 and the internal scattered component I2 in a reflected light pulse from a portion to be measured while distinguishing the surface reflected component I1 and the internal scattered component I2. In a case where the portion to be measured is a forehead, a signal intensity of the internal scattered component I2 to be detected is very small. This is because light of a very small light amount that satisfies the laser safety standards is emitted as described above and scattering and absorption of light by a scalp, a cerebral fluid, a skull bone, gray matter, white matter, and blood are large. Furthermore, a change in signal intensity caused by a change in blood flow amount or component in a blood flow during brain activity is one-several tenth of a signal intensity before the change and is very small. Therefore, in a case where the internal scattered component I2 is detected, the surface reflected component I1, which is several thousands to several tens of thousands of times larger than the internal scattered component to be detected, is removed to a maximum extent.

As described above, when the light emitting device 112 irradiates the user 50 with a light pulse, the surface reflected component I1 and the internal scattered component I2 are generated. Part of the surface reflected component I1 and part of the internal scattered component I2 reach the light receiving device 114. The internal scattered component I2 passes the inside of the user 50 after emission from the light emitting device 112 until the internal scattered component I2 reaches the light receiving device 114. Accordingly, an optical path length of the internal scattered component I2 is longer than an optical path length of the surface reflected component IL Therefore, a timing at which the internal scattered component I2 reaches the light receiving device 114 is later than a timing at which the surface reflected component I1 reaches the light receiving device 114 on average.

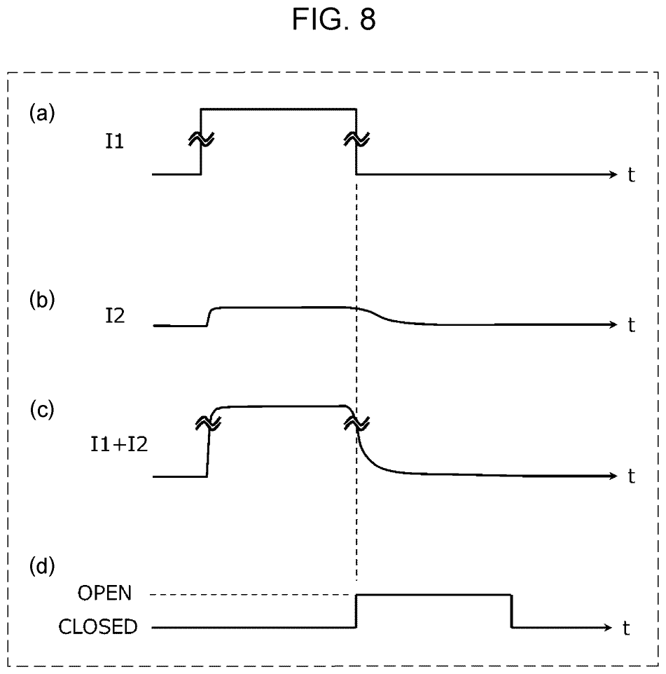

FIG. 8 schematically illustrates a waveform of a light intensity of a reflected light pulse reflected back from the portion to be measured of the user 50 in a case where a rectangular-wave light pulse is emitted from the light emitting device 112. Each horizontal axis represents a time (t). The vertical axis represents an intensity in (a) to (c) of FIG. 8, and represents an OPEN or CLOSED state of the electronic shutter in (d) of FIG. 8. (a) of FIG. 8 illustrates the surface reflected component I1. (b) of FIG. 8 illustrates the internal scattered component I2. (c) of FIG. 8 illustrates a sum of the surface reflected component I1 and the internal scattered component I2. As illustrated in (a) of FIG. 8, the surface reflected component I1 maintains an almost rectangular waveform. On the other hand, the internal scattered component I2 is a combination of light beams of various optical path lengths. Accordingly, as illustrated in (b) of FIG. 8, the internal scattered component I2 exhibits such a characteristic that a rear end of the light pulse has a long tail-like shape. In other words, a falling period of the internal scattered component I2 is longer than a falling period of the surface reflected component I1. To extract the internal scattered component I2 from the optical signal illustrated in (c) of FIG. 8 at a high percentage, exposure of the electronic shutter is started at or after a timing at which the rear end of the surface reflected component I1 reaches the light receiving device 114, as illustrated in (d) of FIG. 8. In other words, exposure is started at or after a time of falling of the waveform of the surface reflected component I1. This shutter timing is adjusted by the control circuit 132.

In a case where the portion to be measured is not flat, a timing of arrival of light differs from one pixel to another of the light receiving device 114. In this case, the shutter timing illustrated in (d) of FIG. 8 may be individually decided for each pixel. For example, assume that a direction orthogonal to the light receiving surface of the light receiving device 114 is a z direction. The control circuit 132 may acquire data indicative of a two-dimensional distribution of a z coordinate on a surface of the portion to be measured and vary the shutter timing from one pixel to another on the basis of this data. This makes it possible to decide an optimal shutter timing at each position even in a case where the surface of the portion to be measured is curved. The data indicative of the two-dimensional distribution of the z coordinate on the surface of the portion to be measured is, for example, acquired by a Time-of-Flight (TOF) technique. In the TOF technique, a period it takes for light emitted by the light emitting device 112 to reach each pixel after being reflected by the portion to be measured is measured. A distance between each pixel and the portion to be measured can be estimated on the basis of a difference between a phase of reflected light detected by the pixel and a phase of the light emitted by the light emitting device 112. In this way, the data indicative of the two-dimensional distribution of the z coordinate on the surface of the portion to be measured can be acquired. The data indicative of the two-dimensional distribution can be acquired before measurement.

In the example illustrated in (a) of FIG. 8, the rear end of the surface reflected component I1 falls vertically. In other words, a period from start to end of falling of the surface reflected component I1 is zero. However, actually, the rear end of the surface reflected component I1 does not fall vertically in some cases. For example, in a case where falling of a waveform of a light pulse emitted from the light emitting device 112 is not completely vertical, in a case where the surface of the portion to be measured has minute irregularities, or in a case where scattering occurs in a surface layer of skin, the rear end of the surface reflected component I1 does not vertically fall. Furthermore, since the user 50 is a non-transparent object, a light amount of the surface reflected component I1 is far larger than a light amount of the internal scattered component I2. Therefore, even in a case where the rear end of the surface reflected component I1 is slightly deviated from a point of vertical falling, there is a possibility that the internal scattered component I2 is buried. Furthermore, a time delay resulting from movement of electrons may occur during a readout period of the electronic shutter. For the above reasons, ideal binary readout such as the one illustrated in (d) of FIG. 8 cannot be sometimes realized. In this case, the control circuit 132 may make a timing of shutter start of the electronic shutter slightly, for example, by approximately 0.5 ns to 5 ns later than a timing immediately after falling of the surface reflected component I1. The control circuit 132 may adjust the emission timing of the light emitting device 112 instead of adjusting the shutter timing of the electronic shutter. In other words, the control circuit 132 may adjust a time difference between the shutter timing of the electronic shutter and the emission timing of the light emitting device 112. In a case where a change in blood flow amount or component in blood in the portion to be measured is measured in a non-contact manner, delaying the shutter timing too much further reduces the internal scattered component I2, which is small from the start. Therefore, the shutter timing may be kept in the vicinity of the rear end of the surface reflected component I1. As described above, a time delay caused by scattering inside the portion to be measured is approximately 4 ns. In this case, a maximum amount of delay of the shutter timing can be approximately 4 ns.

As in the example illustrated in FIG. 7, light pulses may be emitted from the light emitting device 112, and signals may be accumulated by performing exposure for each of the light pulses at shutter timings whose time differences are equal. This amplifies a detected light amount of the internal scattered component I2.

The offset component may be estimated by performing imaging for the same exposure period in a state where no light is emitted by the light emitting device 112 instead of or in addition to disposing a bandpass filter between the user and the light receiving device 114. The estimated offset component is removed by subtraction from a signal detected by each pixel of the light receiving device 114. This makes it possible to remove a dark current component generated on the light receiving device 114.

The internal scattered component I2 includes information on the inside of the user such as cerebral blood flow information. An amount of light absorbed by blood changes in accordance with a temporal change in cerebral blood flow amount of the user As a result, an amount of light detected by the light receiving device 114 increases or decreases accordingly. It is therefore possible to estimate a state of brain activity from the change in cerebral blood flow amount of the user 50 by monitoring the internal scattered component I2.

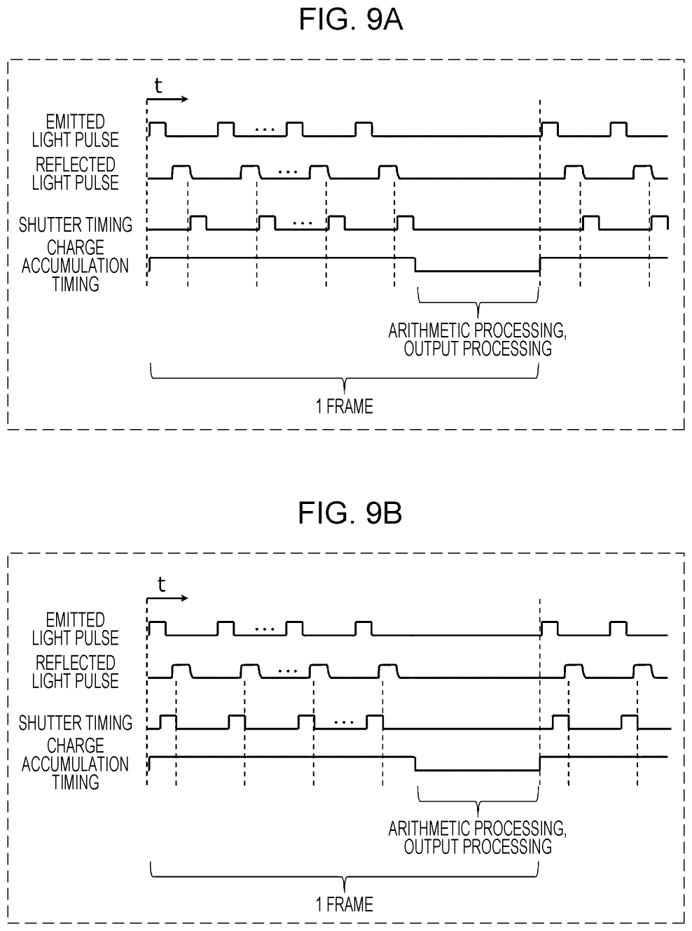

FIG. 9A is a timing diagram illustrating an example of an operation of detecting the internal scattered component I2. In this case, the light emitting device 112 repeatedly emits a light pulse during a one-frame period. The light receiving device 114 opens the electronic shutter during a period where a rear end portion of each reflected light pulse reaches the light receiving device 114. By this operation, the light receiving device 114 accumulates a signal of the internal scattered component I2. After signal accumulation is performed a predetermined number of times, the light receiving device 114 outputs a signal accumulated for each pixel as a detection signal. The output detection signal is processed by the signal processing circuit 134.

As described above, the control circuit 132 repeats the detection operation of causing the light emitting device 112 to emit a light pulse and causing the light receiving device 114 to detect at least a part of a component after start of falling among components of the reflected light pulse and output a detection signal indicative of a spatial distribution of an intensity of an internal scattered component. By such an operation, the signal processing circuit 134 can generate and output distribution data indicative of a spatial distribution of a cerebral blood flow amount in the portion to be measured on the basis of the detection signal that is repeatedly output.

As in the example of FIG. 7, the measurement device 100 may further detect the surface reflected component I1. The surface reflected component I1 includes information on a surface of the user 50. The information on the surface is, for example, information on a blood flow of a face and a scalp.

FIG. 9B is a timing diagram illustrating an example of an operation of detecting the surface reflected component I1. In a case where the surface reflected component I1 is detected, the light receiving device 114 opens the shutter before each reflected light pulse reaches the light receiving device 114 and closes the shutter before the rear end of the reflected light pulse reaches the light receiving device 114. By thus controlling the shutter, it is possible to suppress inclusion of the internal scattered component I2 and increase a proportion of the surface reflected component I1. The timing at which the shutter is closed may be immediately after light reaches the light receiving device 114. This makes it possible to perform signal detection in which a proportion of the surface reflected component I1 having a relatively short optical path length is increased. By acquiring a signal of the surface reflected component I1, it is possible to not only acquire a face image of the user 50, but also estimate a pulse or a degree of oxygenation of a blood flow in a surface layer of skin. In a case where a pulse, that is, a heart rate is estimated from a signal of the surface reflected component I1, the cerebral blood flow sensor 110 functions as the heart rate sensor 120.

The signal of the internal scattered component I2 may be acquired by using light of two wavelengths. For example, a light pulse having a wavelength of 750 nm and a light pulse having a wavelength of 850 nm may be used. This makes it possible to calculate a change in concentration of oxyhemoglobin and a change in concentration of deoxyhemoglobin from changes in amount of detected light of the wavelengths. In a case where the surface reflected component I1 and the internal scattered component I2 are acquired by using two wavelengths, a method of switching four kinds of charge accumulation at a high rate within one frame can be used, for example, as described with reference to FIGS. 5 to 7. By such a method, a temporal deviation of a detection signal can be reduced.

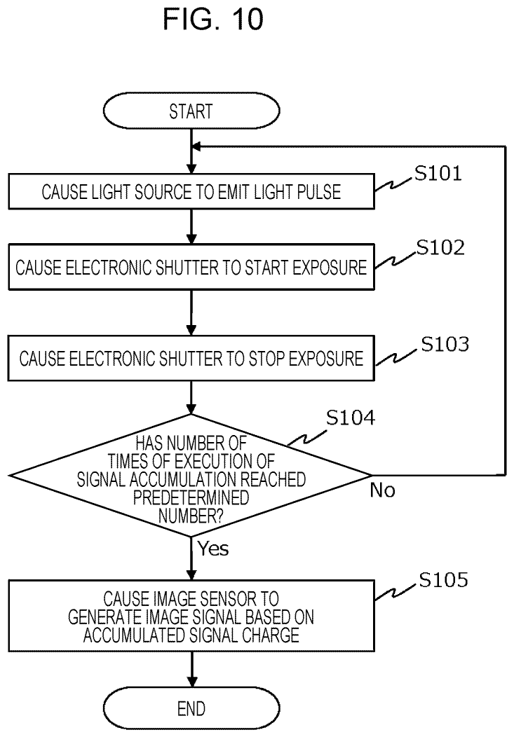

FIG. 10 is a flowchart illustrating an outline of an operation of controlling the light emitting device 112 and the light receiving device 114 by the control circuit 132. The following describes an example of an operation performed in a case where only the internal scattered component I2 is detected by using light of a single wavelength. An operation of detecting the surface reflected component I1 is similar to the operation illustrated in FIG. 10 except for that timings of start and end of exposure relative to an emission timing are earlier. In a case where light of wavelengths is used, the operation illustrated in FIG. 10 is repeated for each wavelength.

In step S101, the control circuit 132 causes the light emitting device 112 to emit a light pulse for a predetermined period. At this time, the electronic shutter of the light receiving device 114 is not performing exposure. The control circuit 132 stops the electronic shutter from performing exposure until a period where a part of the light pulse is reflected by a surface of the forehead of the user 50 and reaches the light receiving device 114 ends. In next step S102, the control circuit 132 causes the electronic shutter to start exposure at a timing at which a part of the light pulse scattered inside the forehead of the user 50 reaches the light receiving device 114. After elapse of a predetermined period, in step S103, the control circuit 132 causes the electronic shutter to stop the exposure. In next step S104, the control circuit 132 determines whether or not the number of times of execution of the signal accumulation has reached a predetermined number. In a case where a result of this determination is No, steps S101 to S103 are repeated until the result of this determination becomes Yes. In a case where the result of the determination in step S104 is Yes, step S105 is performed, in which the control circuit 132 causes the light receiving device 114 to generate and output a signal indicative of an image based on signal charges accumulated in the floating diffusion layers.

By the above operation, a light component scattered inside the measurement target can be detected with high sensitivity. Note that the emission and exposure need not necessarily be performed plural times and are performed plural times as needed.

### 2-5. Example of Signal Processing

Next, an example of signal processing performed by the signal processing circuit 134 is described.

The signal processing circuit 134 generates a cerebral blood flow signal of the user 50 on the basis of a detection signal of each pixel output from the light receiving device 114. The cerebral blood flow signal includes, for example, includes information on an oxyhemoglobin concentration in cerebral blood. The cerebral blood flow signal may further include information on a deoxyhemoglobin concentration or a total hemoglobin concentration, which is a sum of the oxyhemoglobin concentration and the deoxyhemoglobin concentration. The signal processing circuit 134 can obtain change amounts of concentrations of the oxyhemoglobin (HbO₂) and the deoxyhemoglobin (Hb) in blood from initial values by solving predetermined simultaneous equations on the basis of a signal value of the internal scattered component I2 measured for each pixel. The simultaneous equations are, for example, expressed by the following expressions (1) and (2):

$${ε}_{OXY}^{750} Δ {HbO}_{2} + {ε}_{deOXY}^{750} Δ Hb = - ln \frac{{I}_{now}^{750}}{{I}_{ini}^{750}} \qquad ( 1 )$$

$${ε}_{OXY}^{850} Δ {HbO}_{2} + {ε}_{deOXY}^{850} Δ Hb = - ln \frac{{I}_{now}^{850}}{{I}_{ini}^{850}} \qquad ( 2 )$$

where ΔHbO₂ and ΔHb represent change amounts of concentrations of HbO₂ and Hb in the blood from initial values, respectively, ε⁷⁵⁰_{OXY} and ε⁷⁵⁰_{deOXY} represent molar absorption coefficients of HbO₂ and Hb at the wavelength of 750 nm, respectively, ε⁸⁵⁰_{OXY} and ε⁸⁵⁰_{deOXY} represent molar absorption coefficients of HbO₂ and Hb at the wavelength of 850 nm, respectively, I⁷⁵⁰ᵢₙᵢ and I⁷⁵⁰_{now} represent detection intensities of the wavelength of 750 nm at an initial time and a measurement time, respectively, and I⁸⁵⁰ᵢₙᵢ and I⁸⁵⁰_{now} represent detection intensities of the wavelength of 850 nm at an initial time and a measurement time, respectively. The signal processing circuit 134 can calculate, for each pixel, the change amounts ΔHbO₂ and ΔHb of the concentrations of HbO₂ and Hb in the blood from the initial values, for example, on the basis of the expression (1) and (2). In this way, data of two-dimensional distributions of the change amounts of the concentrations of HbO₂ and Hb in the blood in the portion to be measured can be generated.

The signal processing circuit 134 can further calculate a degree of oxygen saturation of hemoglobin. The degree of oxygen saturation is a value indicative of a percentage of hemoglobin in the blood bound to oxygen. The degree of oxygen saturation is defined by the following expression:

degree of oxygen saturation=C(HbO₂)/[C(HbO₂)+C(Hb)]×100 (%)

where C (Hb) is a concentration of the deoxyhemoglobin and C (HbO₂) is a concentration of the oxyhemoglobin.

The living body includes components that absorb red light and near-infrared light in addition to blood. However, a temporal fluctuation in light absorption rate is mainly caused by hemoglobin in arterial blood. Therefore, a degree of oxygen saturation in blood can be measured with high accuracy on the basis of a fluctuation in absorption rate.

Note that the signal processing circuit 134 may calculate only the change amount ΔHbO₂ of the oxyhemoglobin concentration from an initial value. This is because ΔHbO₂ is mainly used in processing for determining an excited state or a relaxed state of the user 50, as described later.

Light that has reached the brain also passes through the scalp and face surface. Accordingly, a fluctuation in blood flow in the scalp and face is also detected. To remove or reduce influence of the fluctuation in blood flow in the scalp and face, the signal processing circuit 134 may perform processing of subtracting the surface reflected component I1 from the internal scattered component I2 detected by the light receiving device 114. This makes it possible to acquire pure cerebral blood flow information excluding blood flow information of the scalp and face. The subtracting method can be, for example, a method of multiplying a signal of the surface reflected component I1 by a coefficient decided in consideration of an optical path length difference and subtracting a value thus obtained from the signal of the internal scattered component I2. This coefficient can be, for example, calculated by simulation or an experiment on the basis of an average of optical constants of general human heads. Such subtracting processing can be easily performed in a case where measurement is performed by using light of a single wavelength by a single measurement device. This is because it is easier to reduce temporal and spatial deviations and achieve matching between characteristics of a scalp blood flow component included in the internal scattered component I2 and characteristics of the surface reflected component I1.

The skull is present between the brain and the scalp. Accordingly, a two-dimensional distribution of a cerebral blood flow and a two-dimensional distribution of a scalp and face blood flow are independent. Therefore, the two-dimensional distribution of the internal scattered component I2 and the two-dimensional distribution of the surface reflected component I1 may be separated on the basis of a signal detected by the light receiving device 114 by using a statistical method such as independent component analysis or principal component analysis.

It is known that there is a close relationship between a change in cerebral blood flow amount or component in blood such as hemoglobin and neural activity of a person. For example, when activity of nerve cells changes in accordance with a change in emotion of a person, a cerebral blood flow amount or a component in blood changes. Accordingly, in a case where biological information such as a change in cerebral blood flow amount or component in blood can be measured, a user's psychological state or physical state can be estimated. The user's psychological state can include, for example, a state such as a mood, an emotion, a health condition, or a sense of temperature. The mood can include, for example, a mood such as a good mood or a bad mood. The emotion can include, for example, an emotion such as a sense of safety, a sense of anxiety, sadness, or anger. The health condition can include, for example, a condition such as a good condition or a fatigued condition. The sense of temperature can include, for example, a sense such as hot, cold, or hot and humid. The psychological state can also include derivatives of these, specifically, indices indicative of a degree of brain activity such as a degree of interest, a degree of proficiency, a level of learning, and a degree of concentration. Furthermore, a physical state such as a degree of fatigue, sleepiness, or a degree of alcohol intoxication may be estimated. In the present specification, such data related to a cerebral blood flow is collectively referred to as “brain activity data”.

### 2-6. Another Example of Cerebral Blood Flow Sensor

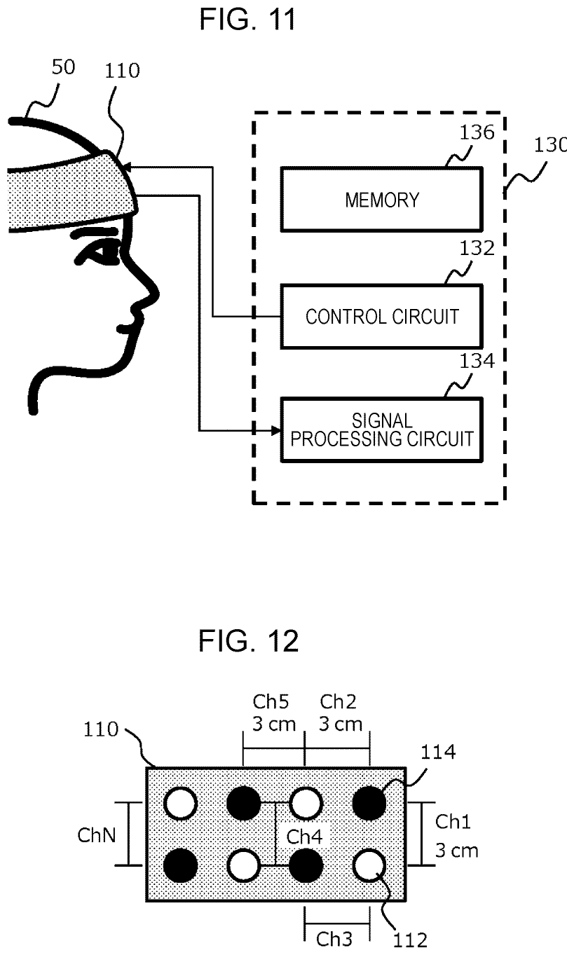

Although the cerebral blood flow sensor 110 is a non-contact type NIRS device in the present embodiment, a contact type NIRS device may be used instead. FIG. 11 illustrates an example in which the cerebral blood flow sensor 110 is a contact type NIRS device. This cerebral blood flow sensor 110 has a band-shaped structure and is wound around the forehead of the user 50. The cerebral blood flow sensor 110 is connected to the processing device 130.

FIG. 12 schematically illustrates an example of a configuration of the cerebral blood flow sensor 110 on a rear side, that is, a side close to the forehead. The cerebral blood flow sensor 110 includes light emitting devices 112 and light receiving devices 114. In the example illustrated in FIG. 12, the light emitting devices 112 and the light receiving devices 114 are arranged in a matrix. Although four light emitting devices 112 and four light receiving devices 114 are provided in this example, the number of light emitting devices 112 and the number of light receiving devices 114 can be any numbers.

In the example illustrated in FIG. 12, each of the light receiving devices 114 is disposed away by 3 cm from a position of an adjacent light emitting device 112 in a lateral or longitudinal direction. A pair of the light emitting device 112 and the light receiving device 114 that are adjacent in the lateral or longitudinal direction is referred to as a “channel (Ch)”. In FIG. 12, channels (Ch1, Ch2, . . . , and CnN) are illustrated. A center-to-center distance between the light emitting device 112 and the light receiving device 114 in each channel is 3 cm in the example illustrated in FIG. 12, but is not limited to this. By thus providing channels as in this example, cerebral blood flow signals at positions can be acquired. The light emitting device 112 and the light receiving device 114 in each channel are controlled by the control circuit 132. The control circuit 132 may measure cerebral blood flow amounts at positions by using all channels or may measure a cerebral blood flow amount by using one or some of the channels. A signal output from each light receiving device 114 is processed by the signal processing circuit 134, and a cerebral blood flow signal and brain activity data are generated.

### 2-6. Specific Example of Emotion Estimation Processing

Next, a specific example of emotion estimation processing performed by the signal processing circuit 134 is described.

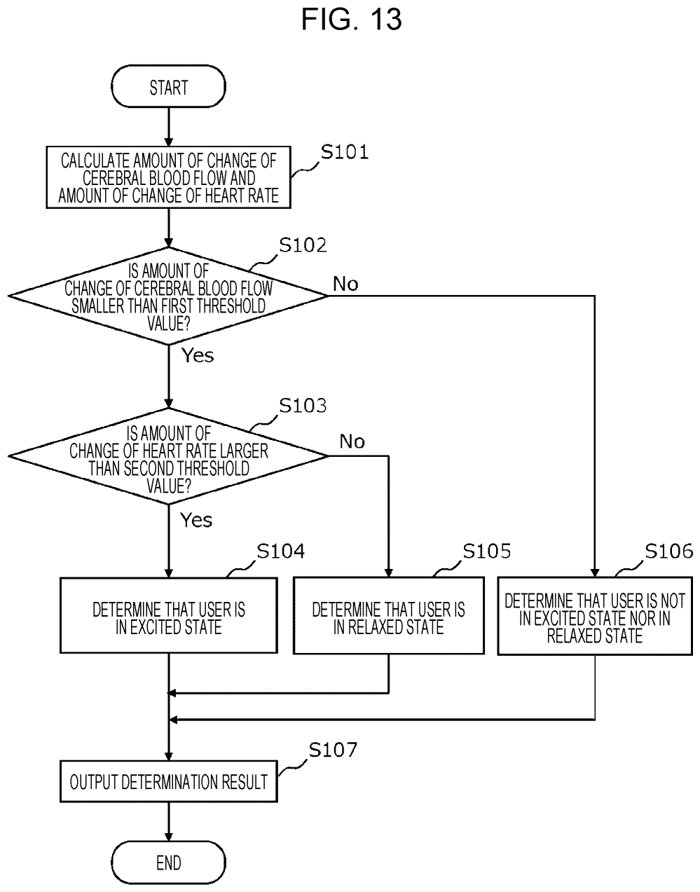

FIG. 13 is a flowchart illustrating an example of the emotion estimation processing performed by the signal processing circuit 134. The signal processing circuit 134 executes the emotion estimation method described with reference to FIGS. 1 and 2. In the example of FIG. 13, the signal processing circuit executes operations in steps S101 to S107 illustrated in FIG. 13.

First, in step S101, the signal processing circuit 134 calculates an amount of change of a cerebral blood flow and an amount of change of a heart rate. The amount of change of the cerebral blood flow can be, for example, an amount of increase of a cerebral blood flow amount of the user 50 measured at a reference time and a measurement time by the cerebral blood flow sensor 110. In the present embodiment, an amount of increase of oxyhemoglobin from the reference time that is calculated on the basis of the above expressions (1) and (2) is used as an amount of change of the cerebral blood flow amount. The amount of change of the heart rate can be, for example, a rate of increase of an average heart rate of the user 50 measured at a reference time and a measurement time by the heart rate sensor 120.

Next, in step S102, the signal processing circuit 134 determines whether or not the amount of change of the cerebral blood flow is smaller than a first threshold value. In a case where the amount of change of the cerebral blood flow is smaller than the first threshold value, step S103 is performed. In a case where the amount of change of the cerebral blood flow is equal to or larger than the first threshold value, step S106 is performed.

In step S103, the signal processing circuit 134 determines whether or not the amount of change of the heart rate is larger than a second threshold value. In a case where the amount of change of the heart rate is larger than the second threshold value, step S104 is performed. In a case where the amount of change of the heart rate is equal to or smaller than the second threshold value, step S105 is performed.

In step S104, the signal processing circuit 134 determines that the user 50 is in an excited state and generates a signal indicating that the user 50 is in an excited state.

In step S105, the signal processing circuit 134 determines that the user 50 is in a relaxed state and generates a signal indicating that the user 50 is in a relaxed state.

In step S106, the signal processing circuit 134 determines that the user 50 is not in an excited state nor in a relaxed state and generates a signal indicating that the user 50 is not in an excited state nor in a relaxed state.

In step S107, the signal processing circuit 134 outputs a signal indicative of a result of determination of a user's emotion performed in step S104, S105, or S106. The signal is sent, for example, to the display device 230. The display device 230 displays an image indicating a user's emotional state on the basis of the signal.

As described above, in the example of FIG. 13, the signal processing circuit 134 determines whether the user 50 is in an excited state or in a relaxed state on the basis of an amount of change of a cerebral blood flow amount and an amount of change of a heart rate of the user 50 and outputs a signal indicative of a result of the determination. By such an operation, whether the user 50 is in an excited state or in a relaxed state as compared with the reference time can be grasped. This enables application such as inducing a desired emotion in the user 50 by controlling the stimulation device 200 in accordance with an emotional state of the user 50.

In the example of FIG. 13, the signal processing circuit 134 determines in step S102 whether or not the amount of change of the cerebral blood flow is smaller than the first threshold value, and the signal processing circuit 134 determines in step S103 whether or not the amount of change of the heart rate is larger than the second threshold value, but this is merely an example. The operations in steps S102 and S103 may be performed in a reverse order. That is, the signal processing circuit 134 may determine whether or not the amount of change of the cerebral blood flow is smaller than the first threshold value after determining whether or not the amount of change of the heart rate is larger than the second threshold value. Alternatively, the determining operations in steps S102 and S103 may be performed concurrently.

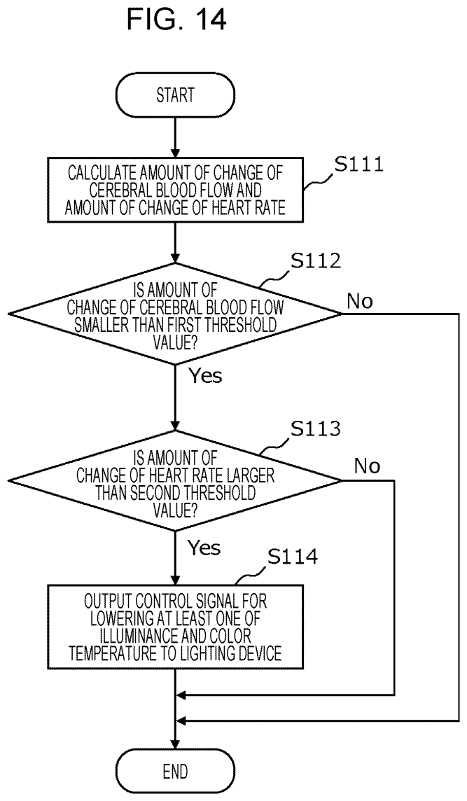

FIG. 14 is a flowchart illustrating another example of the emotion estimation processing performed by the signal processing circuit 134. In this example, in a case where it is estimated that the user 50 is in an excited state, the signal processing circuit 134 generates a control signal for lowering at least one of illuminance or a color temperature of light output from the lighting device 210. This can calm the user's excited state.

First, in step S111, the signal processing circuit 134 calculates an amount of change of a cerebral blood flow and an amount of change of a heart rate as in the earlier example.

Next, in step S112, the signal processing circuit 134 determines whether or not the amount of change of the cerebral blood flow is smaller than the first threshold value. In a case where the amount of change of the cerebral blood flow is less than the first threshold value, step S113 is performed. In a case where the amount of change of the cerebral blood flow is equal to or larger than the first threshold value, it is estimated that the user 50 is not in an excited state, and the operation is finished.

In step S113, the signal processing circuit 134 determines whether or not the amount of change of the heart rate is larger than the second threshold value. In a case where the amount of change of the heart rate is larger than the second threshold value, it is estimated that the user 50 is in an excited state, and step S114 is performed. In a case where the amount of change of the heart rate is equal to or smaller than the second threshold value, it is estimated that the user 50 is not in an excited state, and the operation is finished.

In step S114, the signal processing circuit 134 generates and outputs a control signal for lowering at least one of illuminance or a color temperature of light output from the lighting device 210. The signal is sent to the lighting device 210. The lighting device 210 receives the signal and lowers at least one of the illuminance or the color temperature of the output light. This can calm the excited state of the user 50.

In step S114, the signal processing circuit 134 may output a control signal for increasing at least one of illuminance or a color temperature of light output from the lighting device 210 instead of lowering at least one of the illuminance or the color temperature of the light. By such an operation, the excited state of the user 50 can be maintained.

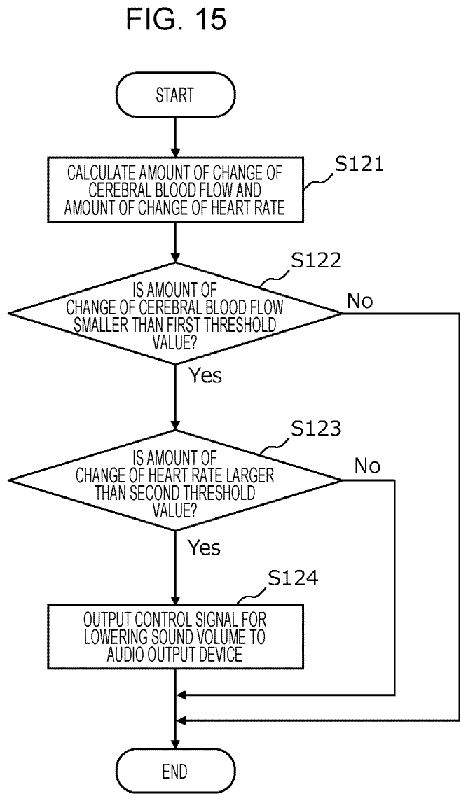

FIG. 15 is a flowchart illustrating still another example of the emotion estimation processing performed by the signal processing circuit 134. In this example, in a case where it is estimated that the user 50 is in an excited state, the signal processing circuit 134 generates a control signal for lowering a sound volume of sound output from the audio output device 220. This can calm the user's excited state.

First, in step S121, the signal processing circuit 134 calculates an amount of change of a cerebral blood flow and an amount of change of a heart rate as in the earlier example.

Next, in step S122, the signal processing circuit 134 determines whether or not the amount of change of the cerebral blood flow is smaller than the first threshold value. In a case where the amount of change of the cerebral blood flow is smaller than the first threshold value, step S123 is performed. In a case where the amount of change of the cerebral blood flow is equal to or larger than the first threshold value, it is estimated that the user 50 is not in an excited state, and the operation is finished.

In step S123, the signal processing circuit 134 determines whether or not the amount of change of the heart rate is larger than the second threshold value. In a case where the amount of change of the heart rate is larger than the second threshold value, it is estimated that the user 50 is in an excited state, and step S124 is performed. In a case where the amount of change of the heart rate is equal to or smaller than the second threshold value, it is estimated that the user 50 is not in an excited state, and the operation is finished.

In step S124, the signal processing circuit 134 generates and outputs a control signal lowering a sound volume of sound output from the audio output device 220. The signal is sent to the audio output device 220. The audio output device 220 receives the signal and lowers the sound volume of the output sound. This can calm the excited state of the user 50.

Note that the user's excited state may be calmed by changing contents of the sound instead of lowering the sound volume of the sound. For example, the excited state may be calmed by control such as changing up-tempo music to slow-tempo music.

In step S124, the signal processing circuit 134 may output a control signal for increasing the sound volume of the sound output from the audio output device 220 instead of lowering the sound volume. By such an operation, the excited state of the user 50 can be maintained.

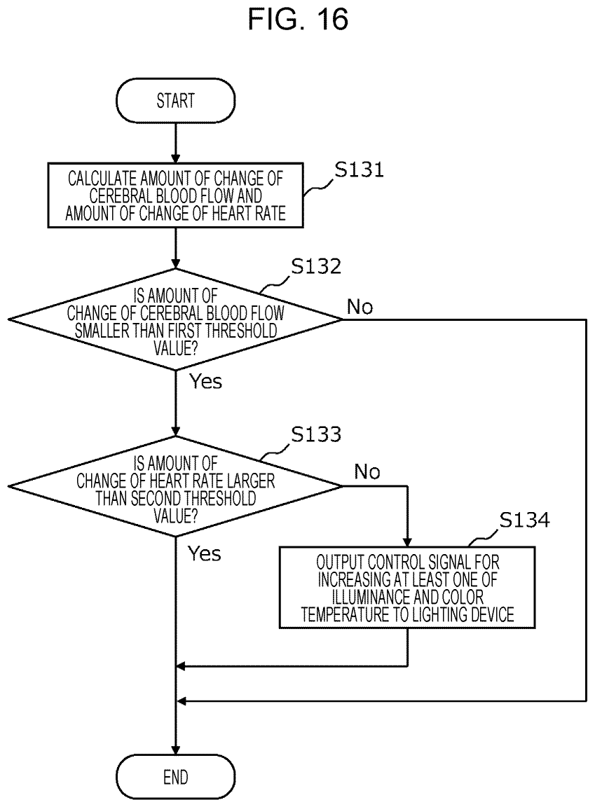

FIG. 16 is a flowchart illustrating still another example of the emotion estimation processing performed by the signal processing circuit 134. In this example, in a case where it is estimated that the user 50 is in a relaxed state, the signal processing circuit 134 generates a control signal for increasing at least one of illuminance or a color temperature of light output from the lighting device 210. This can guide the user to an excited state.

First, in step S131, the signal processing circuit 134 calculates an amount of change of a cerebral blood flow and an amount of change of a heart rate as in the earlier example.

Next, in step S132, the signal processing circuit 134 determines whether or not the amount of change of the cerebral blood flow is smaller than the first threshold value. In a case where the amount of change of the cerebral blood flow is smaller than the first threshold value, step S133 is performed. In a case where the amount of change of the cerebral blood flow is equal to or larger than the first threshold value, it is estimated that the user 50 is not in a relaxed state, and the operation is finished.

In step S133, the signal processing circuit 134 determines whether or not the amount of change of the heart rate is larger than the second threshold value. Note that the second threshold value may be a value different from the second threshold value in the example of FIGS. 14 and 15. In a case where the amount of change of the heart rate is larger than the second threshold value, it is estimated that the user 50 is not in a relaxed state, and the operation is finished. In a case where the amount of change of the heart rate is equal to or smaller than the second threshold value, it is estimated that the user 50 is in a relaxed state, and step S134 is performed.

In step S134, the signal processing circuit 134 generates and outputs a control signal for increasing at least one of illuminance or a color temperature of light output from the lighting device 210. The lighting device 210 receives the signal and increases at least one of the illuminance or the color temperature of the output light. This can guide the user 50 to an excited state.

In step S134, the signal processing circuit 134 may output a control signal for lowering at least one of illuminance or a color temperature of light output from the lighting device 210 instead of increasing at least one of the illuminance or the color temperature of the light. By such an operation, the relaxed state of the user 50 can be maintained.

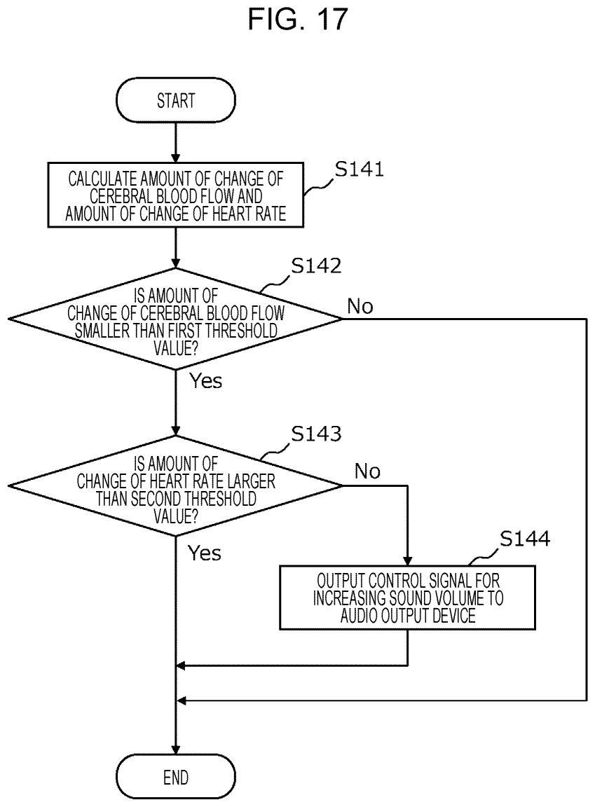

FIG. 17 is a flowchart illustrating still another example of the emotion estimation processing performed by the signal processing circuit 134. In this example, in a case where it is estimated that the user 50 is in a relaxed state, the signal processing circuit 134 generates a control signal for increasing a sound volume of sound output from the audio output device 220. This can guide the user to an excited state.

First, in step S141, the signal processing circuit 134 calculates an amount of change of a cerebral blood flow and an amount of change of a heart rate as in the earlier example.

Next, in step S142, the signal processing circuit 134 determines whether or not the amount of change of the cerebral blood flow is smaller than the first threshold value. In a case where the amount of change of the cerebral blood flow is smaller than the first threshold value, step S143 is performed. In a case where the amount of change of the cerebral blood flow is equal to or larger than the first threshold value, it is estimated that the user 50 is not in a relaxed state, and the operation is finished.

In step S143, the signal processing circuit 134 determines whether or not the amount of change of the heart rate is larger than the second threshold value. Note that this second threshold value may be a value different from the second threshold value in the example of FIGS. 14 and 15. In a case where the amount of change of the heart rate is larger than the second threshold value, it is estimated that the user 50 is not in a relaxed state, and the operation is finished. In a case where the amount of change of the heart rate is equal to or smaller than the second threshold value, it is estimated that the user 50 is in a relaxed state, and step S144 is performed.

In step S144, the signal processing circuit 134 generates and outputs a control signal for increasing a sound volume of sound output from the audio output device 220. The audio output device 220 receives the signal and increases the sound volume of the output sound. This can guide the user 50 to an excited state.

Note that the user may be guided to an excited state by changing contents of the sound instead of increasing the sound volume of the sound. For example, the user may be guided to an excited state by control such as changing slow-tempo music to up-tempo music.

In step S144, the signal processing circuit 134 may output a control signal for lowering the sound volume of the sound output from the audio output device 220 instead of increasing the sound volume. By such an operation, a relaxed state of the user 50 can be maintained.

In the above examples, the signal processing circuit 134 generates a control signal for controlling the lighting device 210 or the audio output device 220 in accordance with an emotional state of the user 50. Such examples are not restrictive, and the signal processing circuit 134 may generate, for example, a control signal for changing contents of an image output from the display device 230 in accordance with an emotional state of the user 50. By changing the contents of the image, the excited state of the user 50 can be further enhanced, the excited state can be calmed, or the user 50 can be guided to a relaxed state.

As described above, according to the present embodiment, the signal processing circuit 134 performs processing for controlling output of the lighting device 210, the audio output device 220, or the display device 230 around the user 50 in a case where it is estimated that the user 50 is in an excited state on the basis of the cerebral blood flow amount and the heart rate. For example, in a case where it is estimated that the user 50 is in an excited state, the signal processing circuit 134 can lower at least one of illuminance or a color temperature of light output from the lighting device 210, lower a sound volume of sound output from the audio output device 220, or cause the audio output device 220 or the display device 230 to output sound or an image that calms the excited state of the user This can calm the excited state of the user 50. Conversely, in a case where it is estimated that the user 50 is in an excited state, the signal processing circuit 134 can increase at least one of illuminance or a color temperature of light output from the lighting device 210, increase a sound volume of sound output from the audio output device 220, or cause the audio output device 220 or the display device 230 to output sound or an image that guides the user 50 to an excited state. This can maintain the excited state of the user

Furthermore, in a case where it is estimated that the user 50 is in a relaxed state, the signal processing circuit 134 can increase at least one of illuminance or a color temperature of light output from the lighting device 210, increase a sound volume of sound output from the audio output device 220, or cause the audio output device 220 or the display device 230 to output sound or an image that guides the user 50 to an excited state. This can induce an excited state in the user 50. Conversely, in a case where it is estimated that the user 50 is in a relaxed state, the signal processing circuit 134 may lower at least one of illuminance or a color temperature of light output from the lighting device 210, lower a sound volume of sound output from the audio output device 220, or cause the audio output device 220 or the display device 230 to output sound or an image that calms an excited state of the user 50. This can maintain the relaxed state of the user 50.

The operation for guiding the user 50 to an excited state by the signal processing circuit 134 may be performed in a case where it is determined that the user 50 is not in an excited state. That is, in a case where the signal processing circuit 134 determines that the amount of change of the cerebral blood flow of the user 50 is larger than the first threshold value or the amount of change of the heart rate of the user 50 is smaller than the second threshold value, the operation for guiding the user 50 to an excited state by the signal processing circuit 134 may be performed.

Data of the image or sound (e.g., music) that guides the user 50 to an excited state and the image or sound that guides the user 50 to a relaxed state may be, for example, stored in a memory of the audio output device 220 or may be, for example, stored in the memory 136 of the measurement device 100. These pieces of data may be stored in a memory of a server connected to the audio output device 220 or the measurement device 100 over a network.

The technique of the present embodiment can be, for example, applied to an application for a mobile device such as a smartphone or a tablet computer. A computer such as a server connected to mobile devices over a network such as the Internet may execute the method according to the present embodiment. The computer may collect a cerebral blood flow signal and a heart rate signal of each user over the network and generate a signal indicating whether or not the user is in an excited state or in a relaxed state. The signal indicating whether or not the user is in an excited state or in a relaxed state is transmitted to a mobile device of the user, and a result of determination can be displayed on a display of the device.

Furthermore, a fragrance releasing device that releases fragrance may be used to guide the user 50 to an excited state or a relaxed state.

In this case, in a case where it is estimated that the user 50 is in an excited state on the basis of the cerebral blood flow amount and the heart rate, the signal processing circuit 134 may perform processing for controlling the fragrance releasing device around the user

For example, in a case where it is estimated that the user 50 is in an excited state, the signal processing circuit 134 may control the fragrance releasing device to release fragrance for inducing a relaxed state such as jasmine, bergamot, rose, lavender, chamomile, cypress, neroli, sandalwood, or cedarwood. This can guide the user 50 who is estimated to be in an excited state to a relaxed state.

Furthermore, in a case where it is estimated that the user 50 is in a relaxed state on the basis of the cerebral blood flow amount and the heart rate, the signal processing circuit 134 may perform processing for controlling the fragrance releasing device around the user 50.

For example, in a case where it is estimated that the user 50 is in a relaxed state, the signal processing circuit 134 may control the fragrance releasing device to release fragrance for inducing an excited state such as peppermint, lemon, rosemary, or lemon grass. This can guide the user 50 who is estimated to be in a relaxed state to an excited state.

This is not restrictive, and in a case where it is estimated that the user 50 is in an excited state on the basis of the cerebral blood flow amount and the heart rate, the signal processing circuit 134 may perform control for releasing fragrance for inducing an excited state in order to maintain the excited state of the user 50. Furthermore, in a case where it is estimated that the user 50 is in a relaxed state on the basis of the cerebral blood flow amount and the heart rate, the signal processing circuit 134 may perform control for releasing fragrance for inducing a relaxed state in order to maintain the relaxed state of the user 50.

Furthermore, control for changing an arousal level of the user 50 may be performed by using an air-conditioning apparatus. Such control is, for example, disclosed in Japanese Unexamined Patent Application Publication No. 2013-012029.

In this case, in a case where it is estimated that the user 50 is in a relaxed state on the basis of the cerebral blood flow amount and the heart rate, the signal processing circuit 134 may perform processing for controlling an air-conditioning apparatus around the user 50.

For example, in a case where it is estimated that the user 50 is in a relaxed state, the signal processing circuit 134 may supply cool air to the user 50 by controlling the air-conditioning apparatus. This may increase an arousal level of the user 50 and can guide the user 50 who is estimated to be in a relaxed state to an excited state. Furthermore, air-conditioning control for adjusting a temperature back to a temperature before the supply of the cool air may be performed after a certain period elapses from the supply of the cool air.

Furthermore, in a case where it is estimated that the user 50 is in an excited state on the basis of the cerebral blood flow amount and the heart rate, the signal processing circuit 134 may perform processing for controlling an air-conditioning apparatus around the user 50.

For example, in a case where it is estimated that the user 50 is in an excited state, the signal processing circuit 134 may supply hot air to the user 50 by controlling the air-conditioning apparatus. This may lower an arousal level of the user 50 and can guide the user 50 who is estimated to be in an excited state to a relaxed state.

Furthermore, in a case where it is estimated that the user 50 is in an excited state on the basis of the cerebral blood flow amount and the heart rate, the signal processing circuit 134 may perform processing for controlling the air-conditioning apparatus around the user 50. For example, in a case where it is estimated that the user 50 is in an excited state, the signal processing circuit 134 generate a 1/f fluctuation air current in a space where the user 50 is present by controlling the air-conditioning apparatus. This can guide the user 50 who is estimated to be in an excited state to a relaxed state. The 1/f fluctuation can mean a fluctuation whose power varies in inverse proportion to a frequency f. It is said that, for example, an interval between human heartbeats, flickering of candle flame, and sound of rain have 1/f fluctuations.

### EXAMPLE

Next, Example in which a user's emotional state was evaluated by the method for estimating an excited state and a relaxed state according to the above embodiment is described.

In the present Example, an experiment of estimating an excited state and a relaxed state was conducted on 50 subjects. A ratio of males to females was 50:50. Each subject listened to exciting music, relaxing music, terrifying music, and white noise (W/N) for comparison while sitting on a chair in a psychology laboratory under a control environment. The exciting music and the relaxing music were music for inducing the respective emotions and were selected beforehand. Specifically, a preliminary survey (the number of surveyed persons: 81) was conducted in which the persons listened to 20 pieces of candidate music, and music that stably induced an excited or relaxed emotion in many persons was selected. As the terrifying music, general music known as music inducing tenor in the field of psychology of music was selected. As the exciting music, music whose score of subjective assessment concerning a question “Did you feel pleasant?” was 4 or more and whose score of subjective assessment concerning a question “Were you aroused?” was 4 or more in a preliminary survey by questionnaire was selected. As the relaxing music, music whose score of subjective assessment concerning a question “Did you feel pleasant?” was 4 or more and whose score of subjective assessment concerning a question “Were you aroused?” was less than 4 in a preliminary survey by questionnaire was selected. The exciting music can be expressed as an emotion located in an upper right quadrant of the Russell's circumplex model, such as aroused, excited, delighted, pleased, or happy. The relaxing music can be expressed as an emotion located in a lower right quadrant of the Russell's circumplex model, such as relaxed, peaceful, serene, calm, at ease, content, or glad. The white noise is a neutral acoustic stimulus that does not induce a specific emotion and was used as a reference for comparison.

The music stimuli were given to each subject by using an amplifier, a player, and a speaker system. A cerebral blood flow was measured by using a contact type NIRS device (OEG-SpO2, Spectratech). A heart rate was measured by using a BIOPAC system (MP160, BIOPAC Systems). As for an experimental protocol, each subject was placed at rest for one minute, was given a music stimulus for two minutes, and was then placed at rest for one minute. An amount of change of a cerebral blood flow and a rate of change of a heart rate at a time when the music stimulus was given were calculated by using a cerebral blood flow and a heart rate during a resting state as a reference.

In an actual use environment, the cerebral blood flow and the heart rate during a resting state can be measured while a user is watching a fixation image in which, for example, a “+” sign is displayed for a certain period (e.g., 1 minute) if a display can be used in the environment. If an acoustic stimulus can be given in the environment, for example, values of a cerebral blood flow and a heart rate measured while a user is listening to an emotion-neutral acoustic stimulus such as white noise or emotion-neutral music can be used as measurement values during a resting state. These are examples, and the emotion-neutral stimulus is not limited to the above examples.

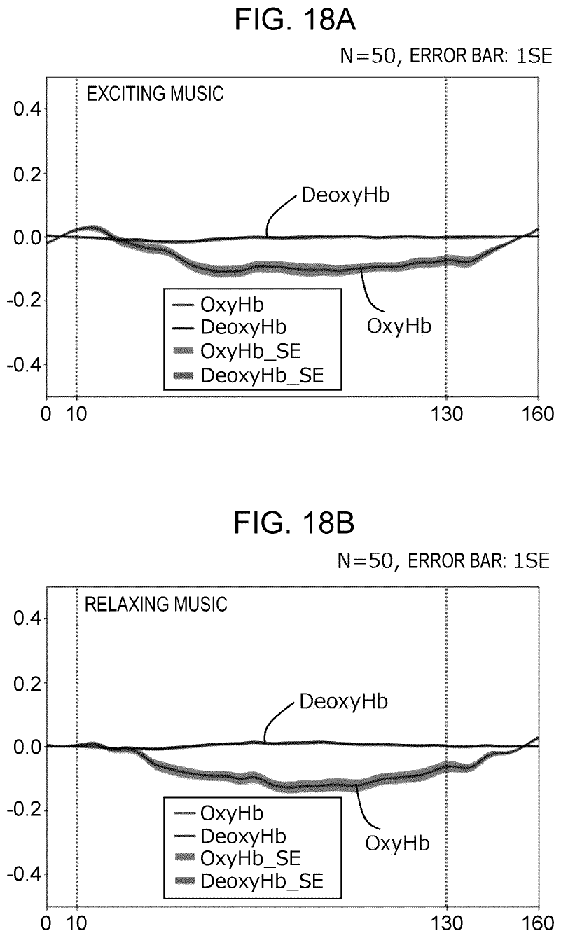

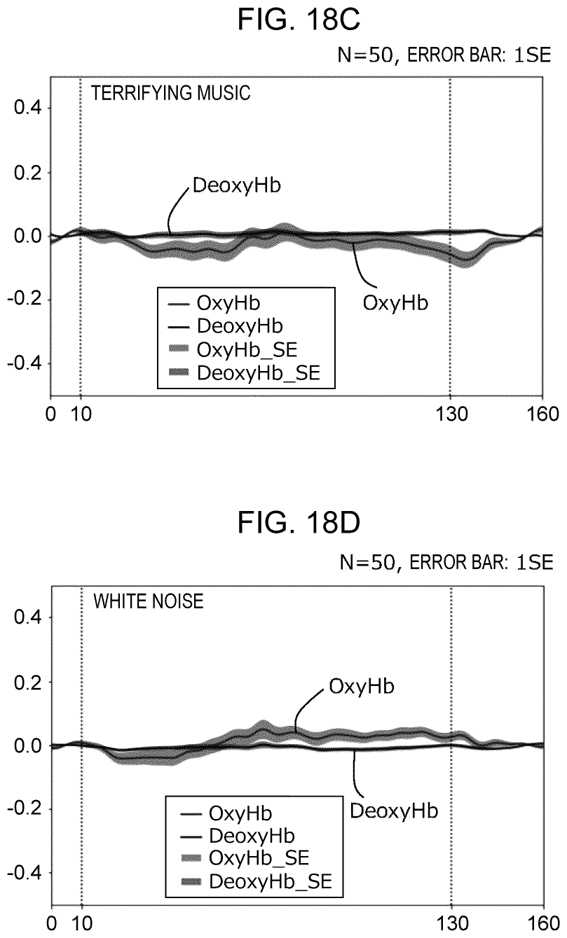

FIGS. 18A to 18D illustrate time-series data of a cerebral blood flow amount (an oxyhemoglobin amount in this example) obtained in a case where the subjects listened to the exciting music, the relaxing music, the terrifying music, and the white noise. The horizontal axis represents a time, and the vertical axis represents an amount of change of a cerebral blood flow. FIGS. 18A to 18D are graphs showing an arithmetic mean of data of the 50 subjects. The error bar represents a standard error 1SE. This result shows that a temporal fluctuation of a cerebral blood flow varies among the exciting music, the relaxing music, the terrifying music, and the white noise. As for the exciting music and the relaxing music, it was confirmed that the cerebral blood flow decreased stably in a statistically-significant amount after elapse of 30 seconds from the start of listening of music until the listening ended. On the other hand, as for the terrifying music and the white noise, a stable change in cerebral blood flow was not observed, and a statistically-significant change was not confirmed.

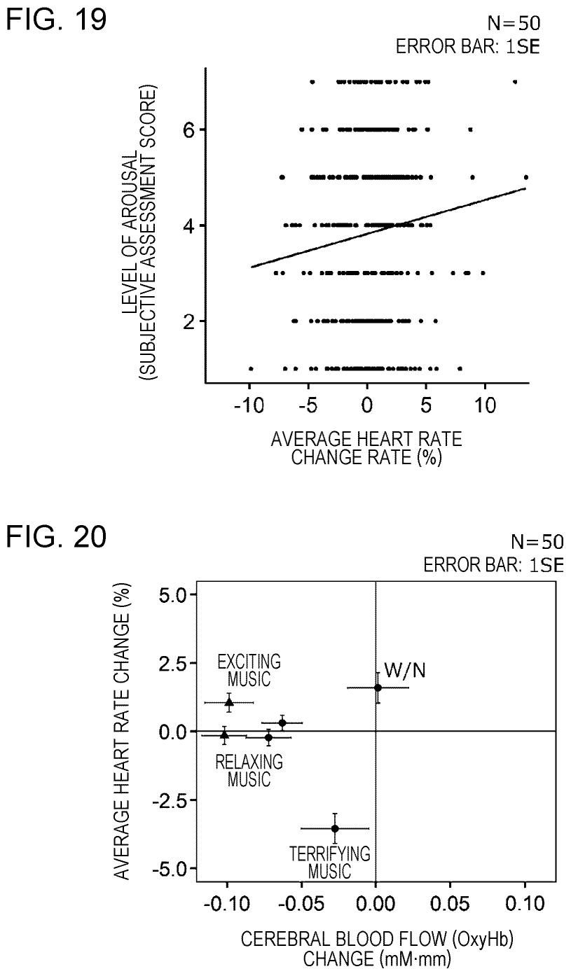

FIG. 19 illustrates a correlation between a change rate of an average heart rate and a subjective assessment score concerning a level of arousal. As illustrated in FIG. 19, a positive correlation was observed between the change rate of the average heart rate and the subjective assessment score concerning a level of arousal. That is, it was confirmed that the change rate of the average heart rate increased as the level of arousal increased.

Regarding a change in cerebral blood flow, the above result revealed that the exciting music and the relaxing music decreased a cerebral blood flow amount. Furthermore, it was revealed that the change rate of the heart rate increased as the level of arousal increased. As described above, an excited state is an emotion located in the upper right quadrant of the Russell's circumplex model, and a relaxed state is an emotion located in the lower right quadrant of the Russell's circumplex model. The upper right quadrant and the lower right quadrant of the Russell's circumplex model are considered to be separate from each other depending on a level of arousal. It is therefore important to consider a change rate of a heart rate, which is correlated to a level of arousal, in estimation of an exited and relaxed states. Therefore, an arithmetic mean of amounts of changes of cerebral blood flows and an arithmetic mean of amounts of changes of average heart rates of the 50 subjects during listening of the four types of music were plotted on a graph whose vertical axis represents an amount of change of a cerebral blood flow and whose horizontal axis represents a change rate of a heart rate, as illustrated in FIG. 20. The error bar represents a standard error 1SE. This result shows that data concerning the exciting music, the relaxing music, the terrifying music, and the white noise are plotted at different positions in FIG. 20. Furthermore, this result shows that no overlapping of error bars indicative of a variation of data is observed for any of the music types and therefore the result is statistically significant.

As is clear from this experiment, an excited state and a relaxed state can be determined with respect to a resting state on the basis of a cerebral blood flow amount and a heart rate with statistical significance, that is, with high accuracy. Specifically, in a case where an amount of change of a cerebral blood flow is smaller than a first threshold value, it can be determined that a user is in an excited state or a relaxed state. Furthermore, in a case where an average heart rate is high in this case, it can be determined that the user is in an excited state, and in a case where an average heart rate is low in this case, it can be determined that the user is in a relaxed state.

The technique of the present disclosure is applicable to a device for determining that a user is in an excited state or a relaxed state on the basis of a cerebral blood flow and a heart rate. The technique of the present disclosure is applicable, for example, to various devices such as a camera, a measurement device, a smartphone, a tablet computer, or a head-mounted device.

What is claimed is:

1. A method executed by a computer, the method comprising:
acquiring an amount of change of a cerebral blood flow of a user from a reference time;
acquiring an amount of change of a heart rate of the user from a reference time; and
outputting a signal indicating that the user is in an excited state in a case where the amount of change of the cerebral blood flow is smaller than a first threshold value and the amount of change of the heart rate is larger than a second threshold value.

2. The method according to claim 1, further comprising outputting a signal indicating that the user is in a relaxed state in a case where the amount of change of the cerebral blood flow is smaller than the first threshold value and the amount of change of the heart rate is smaller than the second threshold value.

3. The method according to claim 1, wherein
the amount of change of the cerebral blood flow is an amount of change of oxyhemoglobin in cerebral blood of the user.

4. The method according to claim 1, wherein
the first threshold value is smaller than 0.

5. The method according to claim 1, wherein
the amount of change of the cerebral blood flow is acquired while the user is viewing or listening to content including sound and/or an image that induces an excited state or a relaxed state in the user; and
the reference time is a time at or before start of the user's viewing or listening of the content.

6. The method according to claim 1, wherein
the signal indicating that the user is in the excited state includes at least one of (i) a signal for controlling output of a lighting device or (ii) a signal for controlling output of an audio output device.

7. The method according to claim 1, further comprising outputting at least one of (i) a control signal for increasing illuminance of a lighting device or (ii) a control signal for causing an audio output device to output sound inducing an excited state in the user in a case where at least one of a condition that the amount of change of the cerebral blood flow is larger than the first threshold value or a condition that the amount of change of the heart rate is smaller than the second threshold value is satisfied.

8. A method executed by a computer, the method comprising:
acquiring an amount of change of a cerebral blood flow of a user from a reference time; and
outputting a signal indicating that the user is in an excited state or a relaxed state in a case where the amount of change of the cerebral blood flow is smaller than a first threshold value.

9. The method according to claim 8, further comprising acquiring an amount of change of a heart rate of the user from a reference time,
wherein the outputting the signal includes outputting a signal indicating that the user is in the excited state in a case where the amount of change of the cerebral blood flow is smaller than the first threshold value and the amount of change of the heart rate is larger than a second threshold value and outputting a signal indicating that the user is in the relaxed state in a case where the amount of change of the cerebral blood flow is smaller than the first threshold value and the amount of change of the heart rate is smaller than the second threshold value.

10. An apparatus comprising:
a cerebral blood flow sensor that measures a cerebral blood flow of a user;
a heart rate sensor that measures a heart rate of the user; and
a signal processing circuit,
wherein the signal processing circuit acquires an amount of change of the cerebral blood flow of the user from a reference time, acquires an amount of change of the heart rate of the user from a reference time, and outputs a signal indicating that the user is an excited state in a case where the amount of change of the cerebral blood flow is smaller than a first threshold value and the amount of change of the heart rate is larger than a second threshold value.

11. An apparatus comprising:
a cerebral blood flow sensor that measures a cerebral blood flow of a user; and
a signal processing circuit,
wherein the signal processing circuit calculates an amount of change of the cerebral blood flow of the user from a reference time on a basis of a signal output from the cerebral blood flow sensor and outputs a signal indicating that the user is in an excited state or a relaxed state in a case where the amount of change of the cerebral blood flow is smaller than a first threshold value.

12. A non-transitory computer-readable recording medium storing a program causing a computer to:
acquire an amount of change of a cerebral blood flow of a user from a reference time;
acquire an amount of change of a heart rate of the user from a reference time; and
output a signal indicating that the user is in an excited state in a case where the amount of change of the cerebral blood flow is smaller than a first threshold value and the amount of change of the heart rate is larger than a second threshold value.

13. A non-transitory computer-readable recording medium storing a program causing a computer to:
acquire an amount of change of a cerebral blood flow of a user from a reference time; and
output a signal indicating that the user is in an excited state or a relaxed state in a case where the amount of change of the cerebral blood flow is smaller than a first threshold value.
